(19) United States
(12) Patent Application Publication
(10) Pub. No.: US 2024/0260892 A1
(43) Pub. Date: Aug. 8, 2024

(54) SYSTEMS AND METHODS FOR SENSOR-BASED, DIGITAL PATIENT ASSESSMENTS

(71) Applicants: Evan Haas, Bucyrus, KS (US); Antony Fuleihan, Orlando, FL (US); Nicholas Theodore, Baltimore, MD (US); Audrey Goo, Irvine, CA (US)

(72) Inventors: Evan Haas, Bucyrus, KS (US); Antony Fuleihan, Orlando, FL (US); Nicholas Theodore, Baltimore, MD (US); Audrey Goo, Irvine, CA (US)

(21) Appl. No.: 18/431,703

(22) Filed: Feb. 2, 2024

Related U.S. Application Data
(60) Provisional application No. 63/442,984, filed on Feb. 2, 2023.

(51) Int. Cl. A61B 5/00; A61B 5/11; G16H 15/00

(52) U.S. Cl. CPC A61B 5/4566; A61B 5/1116; A61B 5/4824; A61B 5/742; G16H 15/00

(57) ABSTRACT

Disclosed are systems and methods that provide a novel framework related to a dynamic spinal assessment tool that provides actionable metrics to guide data-driven, personalized treatment for Adult Spinal Deformity (ASD) and other degenerative spine conditions. The disclosed assessment tool, while worn and/or associated with a patient, can collect physiological patient data for a predetermined period of time, whereby decision-based intelligence software can process the collected data into actionable clinical reports. The dynamically and automatically generated reports, which can be embodied as a digital and/or data structure record of the collected data and/or computational analysis based therefrom, can provide medical professionals (e.g., physicians) with dynamic, patient-specific information for preoperative, intra-operative and/or post-operative stages/procedures.

### CROSS-REFERENCE TO RELATED APPLICATIONS

This application claims the benefit of priority from U.S. Provisional Patent Application No. 63/442,984, filed Feb. 2, 2023, which is incorporated in its entirety herein by reference.

### FIELD OF THE DISCLOSURE

The present disclosure is generally related to a dynamic motion and pain measurement device, and more particularly in some embodiments, to a decision intelligence (DI)-based computerized framework for deterministically monitoring and tracking the movement of a patient as well as the pain thresholds the patient experiences due to such movement.

### SUMMARY OF THE DISCLOSURE

According to some embodiments, as discussed herein, disclosed are systems and methods for a novel computerized framework for tracking the motion of a wearer and the associated physiological signals. According to some embodiments, the disclosed framework can operate as a specifically configured, novel wearable device. In some embodiments, the framework can be implemented within a commercial-off-the-shelf device and/or sensor device, whereby the disclosed framework's installation and/or execution therefrom can provide the novel capabilities discussed herein.

According to some embodiments, the disclosed systems and methods can be utilized for assessing patients experiencing spinal health issues. In some embodiments, it should be understood by those of ordinary skill in the art that while the discussion herein will focus on spinal assessments for patients, it should not be construed as limiting, as the disclosed systems and methods can be utilized for a vast array of other medical and other applications without departing from the scope of the instant disclosure. For example, such medical applications can include, but are not limited to, spine conditions, amyotrophic lateral sclerosis (ALS), Parkinson's, Dementia, Cervical Myelopathy, Stroke, fall risk, fall detection, determining reasons for falls, cancer patients, assessment of mobility, gait rehabilitation, gait training, determining proper mobility aid (walker, cane, braces, and the like), sports related injury, human spinal cord injuries, any neurodegenerative condition, progression of physiological symptoms, hip and/or knee sensors for recovery of non-spinal, orthopedic monitoring, and the like.

Moreover, in some embodiments, as evident from the disclosure herein, domains outside of healthcare may also benefit from the disclosed technology, such as, but not limited to, posture training, sports, strength training, workman's comp. claim investigators, and ergonomic design consultants, and the like, which one of skill in the art would understand would fall within the scope of the disclosed systems and methods.

According to some embodiments, the disclosed device can be worn by a patient and can generate a report for medical professionals (e.g., physicians, for example) that can be leveraged for the determination of the best treatment path for the patient (e.g., steroids, nerve ablations, PT, and the like), and/or the type of surgical intervention and/or surgical planning such as which spinal levels to fuse, what correction angle to use, and the optimal surgical approach to use. In some embodiments, the disclosed framework can automatically leverage the collected patient data to determine the treatment plan, which can be included in the provided report.

As evident from the discussion herein, there are multiple disease states that can benefit from the disclosed monitoring framework. For example, any disease state that has a significant change in mobility or motion could be tracked via the mechanisms disclosed herein. For example, this can include, but is not limited to, spinal conditions, stroke, Parkinson's, and the like. Further, diseases that impact muscular activation but not necessarily mobility or motion can also be analyzed according to some embodiments.

According to some embodiments, a method is disclosed for a DI-based computerized framework for deterministically monitoring and tracking the motion/movement of a patient as well as the pain thresholds the patient experiences subject to such motion/movement. In accordance with some embodiments, the present disclosure provides a non-transitory computer-readable storage medium for carrying out the above-mentioned technical steps of the framework's functionality. The non-transitory computer-readable storage medium has tangibly stored thereon, or tangibly encoded thereon, computer readable instructions that when executed by a device cause at least one processor to perform a method for a DI-based computerized framework for deterministically monitoring and tracking the motion/movement of a patient as well as the pain thresholds the patient experiences subject to such motion/movement.

In accordance with one or more embodiments, a system is provided that includes one or more processors and/or computing devices configured to provide functionality in accordance with such embodiments. In accordance with one or more embodiments, functionality is embodied in steps of a method performed by at least one computing device. In accordance with one or more embodiments, program code (or program logic) executed by a processor(s) of a computing device to implement functionality in accordance with one or more such embodiments is embodied in, by and/or on a non-transitory computer-readable medium.

### DESCRIPTIONS OF THE DRAWINGS

The features, and advantages of the disclosure will be apparent from the following description of embodiments as illustrated in the accompanying drawings, in which reference characters refer to the same parts throughout the various views. The drawings are not necessarily to scale, emphasis instead being placed upon illustrating principles of the disclosure:

FIG. 1 is a block diagram of an example configuration within which the systems and methods disclosed herein could be implemented according to some embodiments of the present disclosure;

FIG. 2 is a block diagram illustrating components of an exemplary system according to some embodiments of the present disclosure;

FIGS. 3A-3D depict a non-limiting exemplary implementation of the disclosed systems and methods according to some embodiments of the present disclosure;

FIG. 4A illustrates an exemplary workflow according to some embodiments of the present disclosure;

FIGS. 4B-4L depict non-limiting example embodiments according to the executable steps of Process 400 of FIG. 4A according to some embodiments of the present disclosure;

FIG. 5 depicts an exemplary implementation of an architecture according to some embodiments of the present disclosure;

FIG. 6 depicts an exemplary implementation of an architecture according to some embodiments of the present disclosure; and

FIG. 7 is a block diagram illustrating a computing device showing an example of a client or server device used in various embodiments of the present disclosure.

### DETAILED DESCRIPTION

The present disclosure will now be described more fully hereinafter with reference to the accompanying drawings, which form a part hereof, and which show, by way of non-limiting illustration, certain example embodiments. Subject matter may, however, be embodied in a variety of different forms and, therefore, covered or claimed subject matter is intended to be construed as not being limited to any example embodiments set forth herein; example embodiments are provided merely to be illustrative. Likewise, a reasonably broad scope for claimed or covered subject matter is intended. Among other things, for example, subject matter may be embodied as methods, devices, components, or systems. Accordingly, embodiments may, for example, take the form of hardware, software, firmware or any combination thereof (other than software per se). The following detailed description is, therefore, not intended to be taken in a limiting sense.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

In general, terminology may be understood at least in part from usage in context. For example, terms, such as “and,” “or,” or “and/or,” as used herein may include a variety of meanings that may depend at least in part upon the context in which such terms are used. Typically, “or” if used to associate a list, such as A, B or C, is intended to mean A, B, and C, here used in the inclusive sense, as well as A, B or C, here used in the exclusive sense. In addition, the term “one or more” as used herein, depending at least in part upon context, may be used to describe any feature, structure, or characteristic in a singular sense or may be used to describe combinations of features, structures or characteristics in a plural sense. Similarly, terms, such as “a,” “an,” or “the,” again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

The present disclosure is described below with reference to block diagrams and operational illustrations of methods and devices. It is understood that each block of the block diagrams or operational illustrations, and combinations of blocks in the block diagrams or operational illustrations, can be implemented by means of analog or digital hardware and computer program instructions. These computer program instructions can be provided to a processor of a general purpose computer to alter its function as detailed herein, a special purpose computer, ASIC, or other programmable data processing apparatus, such that the instructions, which execute via the processor of the computer or other programmable data processing apparatus, implement the functions/acts specified in the block diagrams or operational block or blocks. In some alternate implementations, the functions/acts noted in the blocks can occur out of the order noted in the operational illustrations. For example, two blocks shown in succession can in fact be executed substantially concurrently or the blocks can sometimes be executed in the reverse order, depending upon the functionality/acts involved.

For the purposes of this disclosure a non-transitory computer readable medium (or computer-readable storage medium/media) stores computer data, which data can include computer program code (or computer-executable instructions) that is executable by a computer, in machine readable form. By way of example, and not limitation, a computer readable medium may include computer readable storage media, for tangible or fixed storage of data, or communication media for transient interpretation of code-containing signals. Computer readable storage media, as used herein, refers to physical or tangible storage (as opposed to signals) and includes without limitation volatile and non-volatile, removable and non-removable media implemented in any method or technology for the tangible storage of information such as computer-readable instructions, data structures, program modules or other data. Computer readable storage media includes, but is not limited to, RAM, ROM, EPROM, EEPROM, flash memory or other solid state memory technology, optical storage, cloud storage, magnetic storage devices, or any other physical or material medium which can be used to tangibly store the desired information or data or instructions and which can be accessed by a computer or processor.

For the purposes of this disclosure the term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

For the purposes of this disclosure a “network” should be understood to refer to a network that may couple devices so that communications may be exchanged, such as between a server and a client device or other types of devices, including between wireless devices coupled via a wireless network, for example. A network may also include mass storage, such as network attached storage (NAS), a storage area network (SAN), a content delivery network (CDN) or other forms of computer or machine-readable media, for example. A network may include the Internet, one or more local area networks (LANs), one or more wide area networks (WANs), wire-line type connections, wireless type connections, cellular or any combination thereof. Likewise, sub-networks, which may employ differing architectures or may be compliant or compatible with differing protocols, may interoperate within a larger network.

For purposes of this disclosure, a “wireless network” should be understood to couple client devices with a network. A wireless network may employ stand-alone ad-hoc networks, mesh networks, Wireless LAN (WLAN) networks, cellular networks, or the like. A wireless network may further employ a plurality of network access technologies, including Wi-Fi, Long Term Evolution (LTE), WLAN, Wireless Router mesh, or 2nd, 3rd, 4^{th} or 5^{th} generation (2G, 3G, 4G or 5G) cellular technology, mobile edge computing (MEC), Bluetooth, 802.11b/g/n, or the like. Network access technologies may enable wide area coverage for devices, such as client devices with varying degrees of mobility, for example.

In short, a wireless network may include virtually any type of wireless communication mechanism by which signals may be communicated between devices, such as a client device or a computing device, between or within a network, or the like.

A computing device may be capable of sending or receiving signals, such as via a wired or wireless network, or may be capable of processing or storing signals, such as in memory as physical memory states, and may, therefore, operate as a server. Thus, devices capable of operating as a server may include, as examples, dedicated rack-mounted servers, desktop computers, laptop computers, set top boxes, integrated devices combining various features, such as two or more features of the foregoing devices, or the like.

For purposes of this disclosure, a client (or user, entity, subscriber or customer) device may include a computing device capable of sending or receiving signals, such as via a wired or a wireless network. A client device may, for example, include a desktop computer or a portable device, such as a cellular telephone, a smart phone, a display pager, a radio frequency (RF) device, an infrared (IR) device a Near Field Communication (NFC) device, a Personal Digital Assistant (PDA), a handheld computer, a tablet computer, a phablet, a laptop computer, a set top box, a wearable computer, smart watch, an integrated or distributed device combining various features, such as features of the forgoing devices, or the like.

A client device may vary in terms of capabilities or features. Claimed subject matter is intended to cover a wide range of potential variations, such as a web-enabled client device or previously mentioned devices may include a high-resolution screen (HD or 4K for example), one or more physical or virtual keyboards, mass storage, one or more accelerometers, one or more gyroscopes, one or more magnetometers, one or more barometers, global positioning system (GPS) or other location-identifying type capability, or a display with a high degree of functionality, such as a touch-sensitive color 2D or 3D display, for example.

Certain embodiments and principles will be discussed in more detail with reference to the figures. According to some embodiments, as discussed herein, the disclosed systems and methods provide a dynamic spinal assessment tool that provides actionable metrics to guide data-driven, personalized treatment for Adult Spinal Deformity (ASD) and other degenerative spine conditions. As discussed above, the disclosed assessment tool, referred to as a framework, can operate as a specifically configured, novel wearable device. In some embodiments, the tool/framework can be implemented within an existing device, whereby the disclosed framework's installation and/or execution therefrom can provide the novel capabilities discussed herein.

According to some embodiments, the disclosed framework can collect physiological patient data for a predetermined period of time (e.g., a 48-hour period, for example), whereby DI-based intelligence engines, modules, software and/or algorithms can process the collected data into actionable clinical reports. As discussed below, in some embodiments, the dynamically and automatically generated report, which can be embodied as a digital and/or data structure record of the collected data and/or computational analysis based therefrom, can provide medical professionals (e.g., physicians) with dynamic, patient-specific information to inform their treatment planning and better communicate with their patients. In some embodiments, the preoperative planning provided herein can further be leveraged for a post-operative period, and in some embodiments, during a procedure (e.g., intra-operate, where monitoring how effective a treatment is as it is being applied/performed can be performed).

According to some embodiments, the disclosed framework can involve three (3) components: a wearable sensor array, an interactable application and a proprietary algorithm. In some embodiments, the wearable array (interchangeably referred to as a device, sensor device and sensor, as discussed herein) can include proprietary sensors that can be placed along key landmarks on a patient's body to capture positional data throughout their activities and while they sleep.

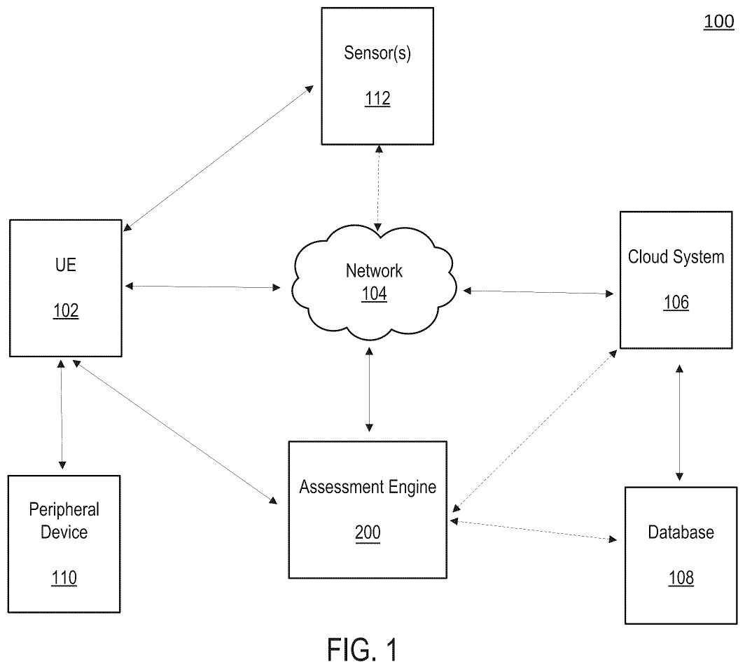

In some embodiments, the disclosed interactable application can collect and/or report metrics and indications related to the patient's movements/motion, which can be electronically provided to an observing medical professional. In some embodiments, for example, the application can execute on a user's device that is communicatively connected to the wearable sensor—for example, the application can execute on a user's smart phone and/or other type of wearable device (e.g., a smart watch), as discussed below in relation to at least FIG. 1. Accordingly, in some embodiments, usage of the user's device and/or wearable device can enable physiological parameter tracking, and electronic transmission and reception of current (e.g., live or real-time, for example) data for monitoring and prompting inputs.

In some embodiments, a user's device that is communicatively connected to the wearable sensor(s) may transmit signals that lead to a change in the user's device and/or wearable device. In some embodiments, this signal may be instructions to start, stop, and/or alter data collection. In some embodiments, this signal may be based on the motion or movement derived from internal sensors such as accelerometers or gyroscopes of the user's device. In some embodiments, data from the user's device may be added either in real-time or after collection to the data collected from the wearable system and used in the analysis of the collection. In some embodiments, data from the wearable sensor(s) may transmit data to the user's device to process, transfer, upload, and/or display data collected from the system.

In some embodiments, the disclosed algorithm, as discussed in more detail below, can execute by processing large amounts of critical information collected over a predetermined period of time (e.g., 48 hours, for example). As provided below, in some embodiments, execution of the algorithm can enable the transformation and output of the collected data into an interactive, interpreted and integrated clinical report for physicians to quickly interact with prior to planning treatments and talking to their patients. In some embodiments, the clinical report can be any type of file and/or displayable output, which can include, but is not limited to, an image, video, simulation, augmented reality (AR) display, virtual reality (VR) display, mixed reality (MR), extended reality (XR) display, rendering, 3D prints, text, multimedia, audio, and the like, or some combination thereof. In some embodiments, the same files and/or displayable output can be incorporated in other methods of the system besides the report. In some embodiments, the system can be combined with a MR/AR/VR simulation in order to live monitor the patient through activities, provide tasks in a controlled setting, etc.

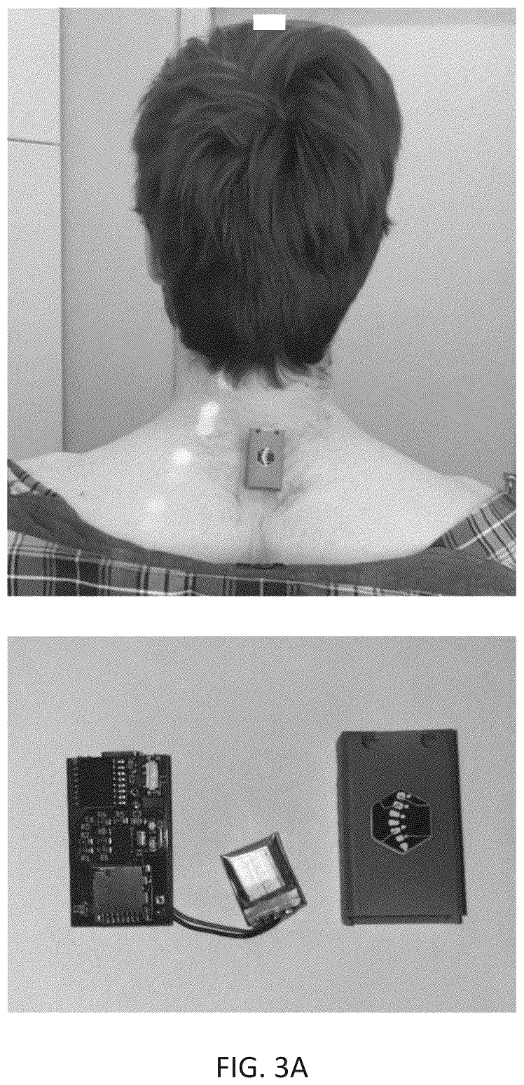

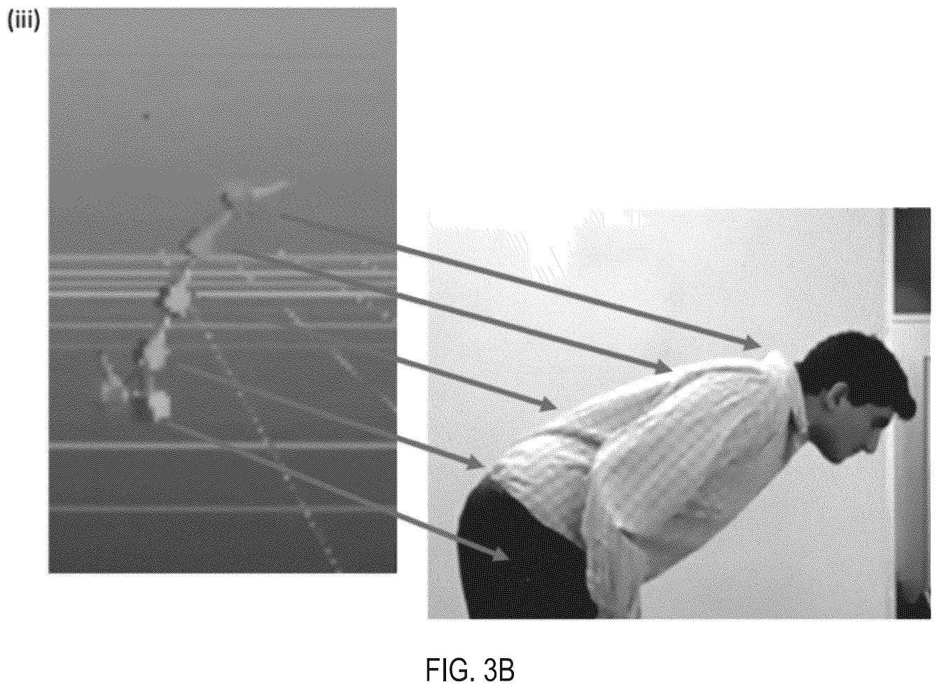

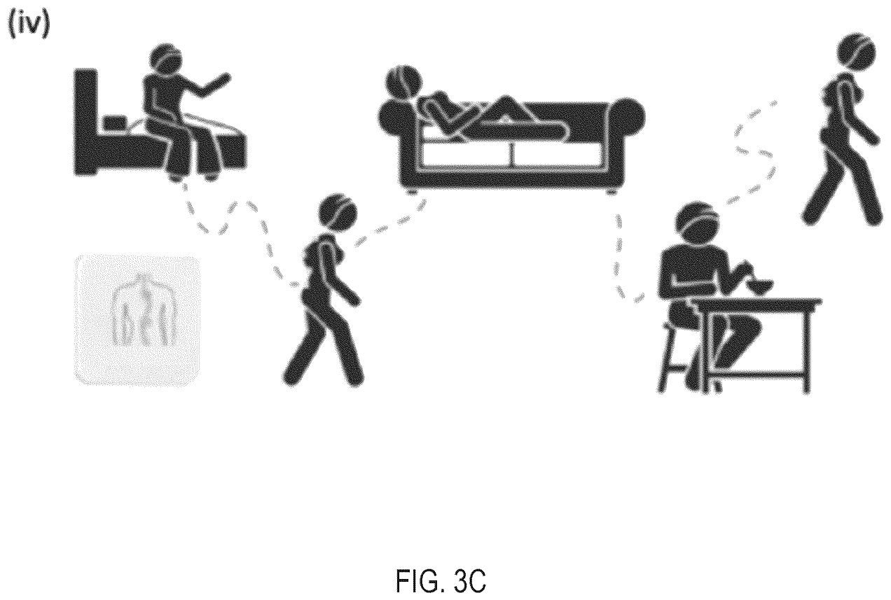

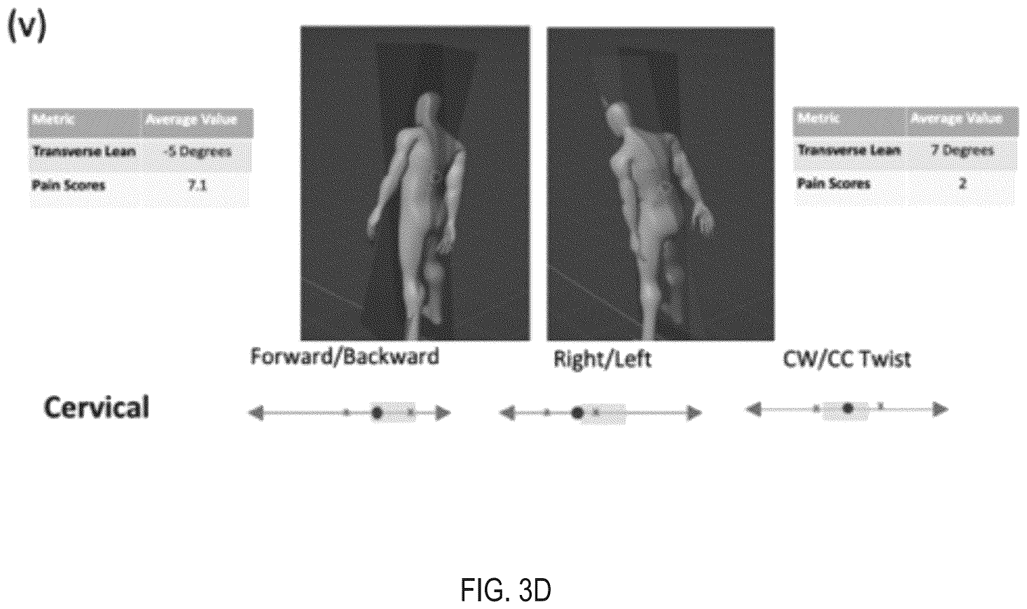

For example, as depicted in FIGS. 3A-3D, respectively provided is a non-limiting implementation of the disclosed systems and methods according to some non-limiting example embodiments. For example, as depicted in FIG. 3A, provided are (i) an image of a prototype sensor on a patient, and (ii) an example of a circuit board and battery (with a depicted ruler for scale). In FIG. 3B, provided is (iii) an image of spinal monitoring example with an example sensor array tracking movement. In FIG. 3C, provided is (iv) an example model image of a patch, as disclosed herein, with illustrated example tracking of daily activities of a patient. And, in FIG. 3D, provided is (v) an example snippet of a clinical report that can be produced via the disclosed data analysis.

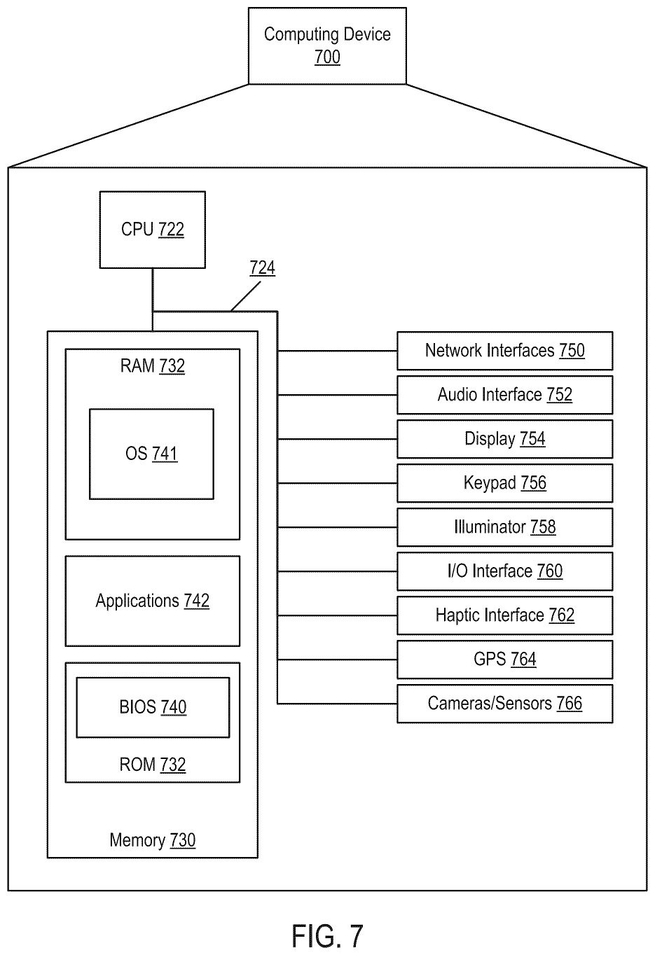

With reference to FIG. 1, system 100 is depicted, which according to some embodiments, can include user equipment (UE) 102 (e.g., a user device, as mentioned above and discussed below in relation to FIG. 7), sensor(s) 112, peripheral device 110, network 104, cloud system 106, database 108 and assessment engine 200. It should be understood that while system 100 is depicted as including such components, it should not be construed as limiting, as one of ordinary skill in the art would readily understand that varying numbers of UEs, peripheral devices, sensors, cloud systems, databases and/or networks can be utilized without departing from the scope of the instant disclosure; however, for purposes of explanation, system 100 is discussed in relation to the example depiction in FIG. 1.

According to some embodiments, UE 102 can be any type of device, such as, but not limited to, a mobile phone, tablet, laptop, sensor, wearable device, wearable camera, wearable clothing, a patch, Internet of Things (IoT) device, autonomous machine, and any other type of modern device. In some embodiments, UE 102 can be a device associated with an individual (or set of individuals) for which motion monitoring services are being provided. In some embodiments, UE 102 may correspond to a reflective marker in which movement data may be tracked via an imaging device not shown.

In some embodiments, UE 102 (and/or peripheral device 110) can provide and/or be connected to a display where a pain and/or motion tracking interface can be provided, which as provided below, can enable the display of data as it is collected, after predetermined intervals of collection and/or after the report is output (e.g., to display the generated report).

In some embodiments, peripheral device 110 can be connected to UE 102, and can be any type of peripheral device, such as, but not limited to, a wearable device (e.g., smart watch), printer, speaker, sensor, neurostimulator, electrical stimulator, and the like. In some embodiments, peripheral device 110 can be any type of device that is connectable or couplable to UE 102 via any type of known or to be known pairing or connection mechanism, including, but not limited to, Bluetooth™, Bluetooth Low Energy (BLE), NFC, WiFi, and the like.

According to some embodiments, a sensor 112 can correspond to sensors associated with a device, clothing, patch and/or any other type of housing or configuration where a sensor can be associated therewith. In some embodiments, UE 102 can have associated therewith a plurality of sensors 112 to collect data from a user. By way of a non-limiting example, the sensors 112 can include the sensors on UE 102 (e.g., smart phone) and/or peripheral device 110 (e.g., a paired smart watch). For example, sensors 112 may be, but are not limited to, an accelerometer or gyroscope that track a patient's movement. For example, an accelerometer may measure acceleration, which is the rate of change of the velocity of an object in meters per second squared (m/s²) or in G-forces (g). Thus, for example, the collected sensor data may indicate a patient's movements, breathing, restlessness, twitches, pauses or other detected movements and/or non-movements that may be common during a performance of a task. In some embodiments, sensors 112 also may track and/or collect x, y, z coordinates of the user and/or UE 102 in order to detect the movements of the user.

According to some embodiments, sensors 112 may be specifically configured for the positional placement respective to a user. For example, a sensor 112 may be situated on an extremity of a user (e.g., arm or leg) and/or may be configured on a user's torso (e.g., a body camera, such as, for example, a chest-worn, hand-worn, foot-worn and/or head/helmet-worn camera, for example). Such sensors 112 can be affixed to the user via the use of bands, adhesives, straps, and the like, or some combination thereof. For example, a sensor can be a fabric wristband (or other type of material/clothing) that has contrast points for detection by an imaging modality (e.g., imaging device, for example, and/or a camera associated with UE 102, for example). Some embodiments provide a garment with built in or coupled sensors.

According to some embodiments, one or more of the sensors 112 may include any type of known or to be known type of sensor (and/or sensors or sensor array), such as, but not limited to, a temperature sensor, a thermal gradient sensor, a barometer, an altimeter, an accelerometer, a gyroscope, a magnetometer, a humidity sensor, a an inclinometer, an oximeter, a colorimetric monitor, a sweat analyte sensor, a galvanic skin response sensor, an interfacial pressure sensor, a force sensing resistor, a capacitive sensor, a flow sensor, a stretch sensor, flex resistor, strain sensor, temperature sensor, fiber optic shape sensor and/or interrogator, ultrasound, pulse-echo sensor, a microphone, and the like, and/or any combination thereof. One or more of the sensors 112 can include, but are not limited to, an inertial measurement unit (IMU), electromyography (EMG), Photoplethysmography (PPG), electrocardiography (EKG), Pulse Oximeter, Bioimpedance, and the like.

According to some embodiments, sensors 112 may be integrated into the operation of the UE 102 in order to monitor the status of a user. In some embodiments, some or all of the data acquired by the sensors 112 may be used to train a machine learning and/or artificial intelligence (ML/AI) algorithm used by the UE 102 and/or artificial intelligence to control the UE 102 or for other desired uses. According to some embodiments, such ML/AI can include, but are not limited to, computer vision, neural network analysis, regressions, graph networks and the like, as discussed below.

In some embodiments, the sensors 112 can be positioned at particular positions (or sub-locations) on the user/patient (e.g., along the spinal column, for example, at predetermined intervals. Such sensors can enable the tracking of positions, movements and/or non-activity of a user, as discussed herein.

In some embodiments, sensors 112 can be connected to other sensors located at a location (e.g., a building, room, structure, and/or any other type of definable area). Such sensors can further enable tracking of a user's movements, and such sensors can be, but are not limited to, cameras, motion detectors, door and window contacts, heat and smoke detectors, passive infrared (PIR) sensors, time-of-flight (ToF) sensors, and the like. In some embodiments, the sensors can be associated with devices associated with the location of system 100, such as, for example, lights, smart locks, garage doors, smart appliances (e.g., thermostats, refrigerators, televisions, personal assistants (e.g., Alexa®, Nest®, for example)), smart phones, smart watches, exoskeletons, or other wearables, tablets, personal computers, and the like, and/or some combination thereof.

In some embodiments, network 104 can be any type of network, such as, but not limited to, a wireless network, cellular network, the Internet, and the like (as discussed above). Network 104 facilitates connectivity of the components of system 100, as illustrated in FIG. 1.

According to some embodiments, cloud system 106 may be any type of cloud operating platform and/or network based system upon which applications, operations, and/or other forms of network resources may be located. For example, system 106 may be a service and/or health provider, and/or network provider from where services and/or applications may be accessed, sourced or executed from. For example, system 106 can represent the cloud-based architecture associated with a healthcare provider, which has associated network resources hosted on the internet or private network (e.g., network 104), which enables (via engine 200) the patient monitoring and management discussed herein.

In some embodiments, cloud system 106 may be a private cloud, where access is restricted by isolating the network such as preventing external access, or by using encryption to limit access to only authorized users. Alternatively, cloud system 106 may be a public cloud where access is widely available via the internet. A public cloud may not be secured or may be include limited healthcare features.

In some embodiments, cloud system 106 may include a server(s) and/or a database of information which is accessible over network 104. In some embodiments, a database 108 of cloud system 106 may store a dataset of data and metadata associated with local and/or network information related to a user(s) of UE 102/device 110 and the UE 102/device 110, sensors 112, imaging device 114, and the services and applications provided by cloud system 106 and/or assessment engine 200.

In some embodiments, for example, cloud system 106 can provide a private/proprietary management platform, whereby engine 200, discussed infra, corresponds to the novel functionality system 106 enables, hosts and provides to a network 104 and other devices/platforms operating thereon.

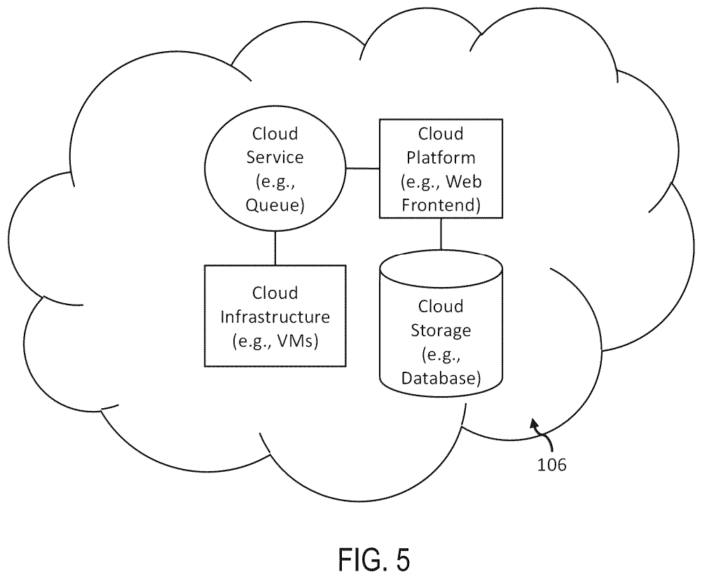

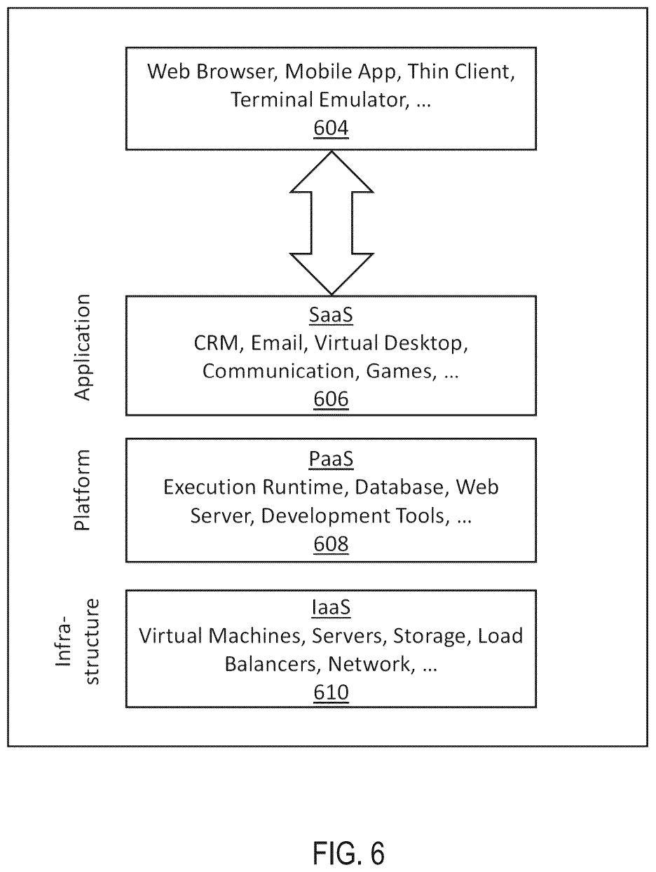

Turning to FIGS. 5-6, in some embodiments, the exemplary computer-based systems/platforms, the exemplary computer-based devices, and/or the exemplary computer-based components of the present disclosure may be specifically configured to operate in a cloud computing/architecture 106 such as, but not limiting to: infrastructure a service (IaaS) 610, platform as a service (PaaS) 608, and/or software as a service (SaaS) 606 using a web browser, mobile app, thin client, terminal emulator or other endpoint 604. FIGS. 5-6 illustrate schematics of non-limiting implementations of the cloud computing/architecture(s) in which the exemplary computer-based systems for administrative customizations and control of network-hosted APIs of the present disclosure may be specifically configured to operate.

Turning back to FIG. 1, according to some embodiments, database 108 may correspond to a data storage for a platform (e.g., a network hosted platform, such as cloud system 106, as discussed supra) or a plurality of platforms. Database 108 may receive storage instructions/requests from, for example, engine 200 (and associated microservices), which may be in any type of known or to be known format, such as, for example, standard query language (SQL). According to some embodiments, database 108 may correspond to any type of known or to be known type of storage, such as, but not limited to a, look-up table (LUT), distributed ledger of a distributed network, and the like.

Assessment engine 200, as discussed above and further below in more detail, can include components for the disclosed functionality. According to some embodiments, assessment engine 200 may be a special purpose machine or processor, and can be hosted by a device on network 104, within cloud system 106 and/or on UE 102 (and/or sensors 112 and/or peripheral device 110). In some embodiments, engine 200 may be hosted by a server and/or set of servers associated with cloud system 106.

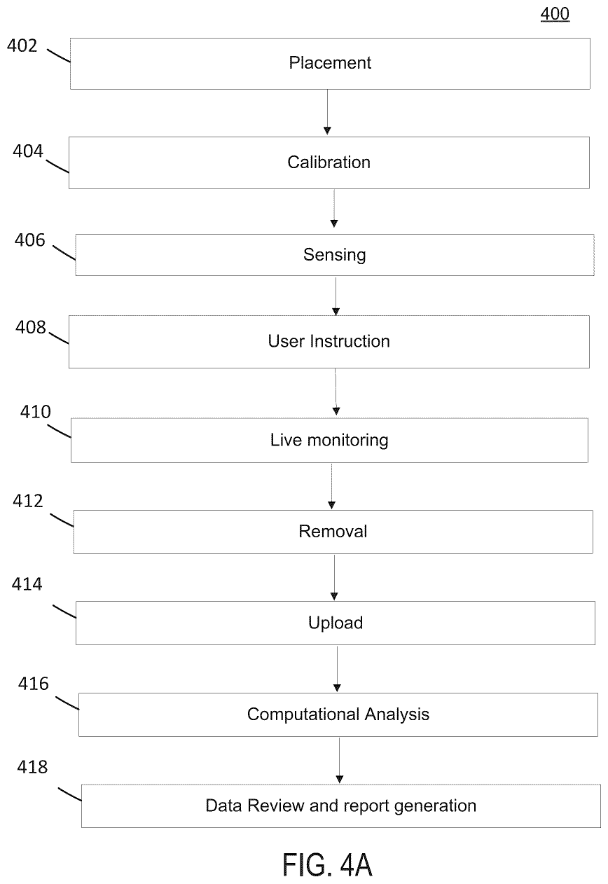

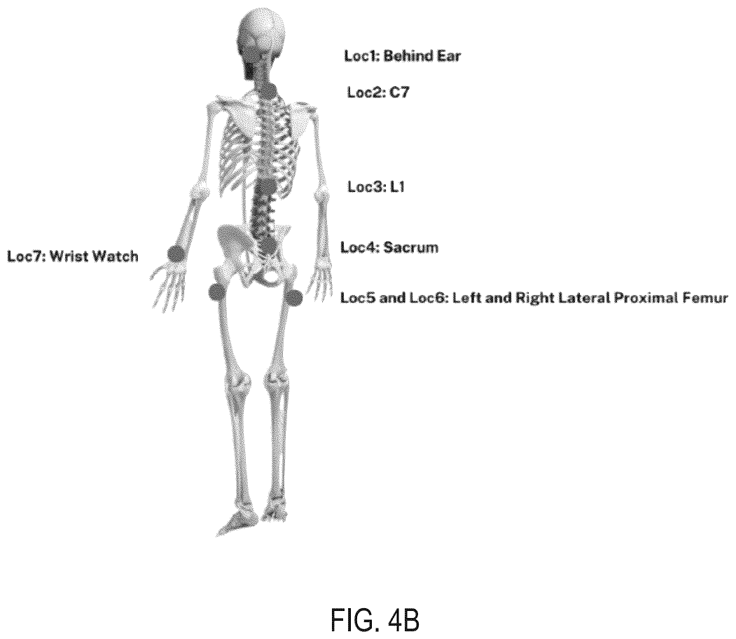

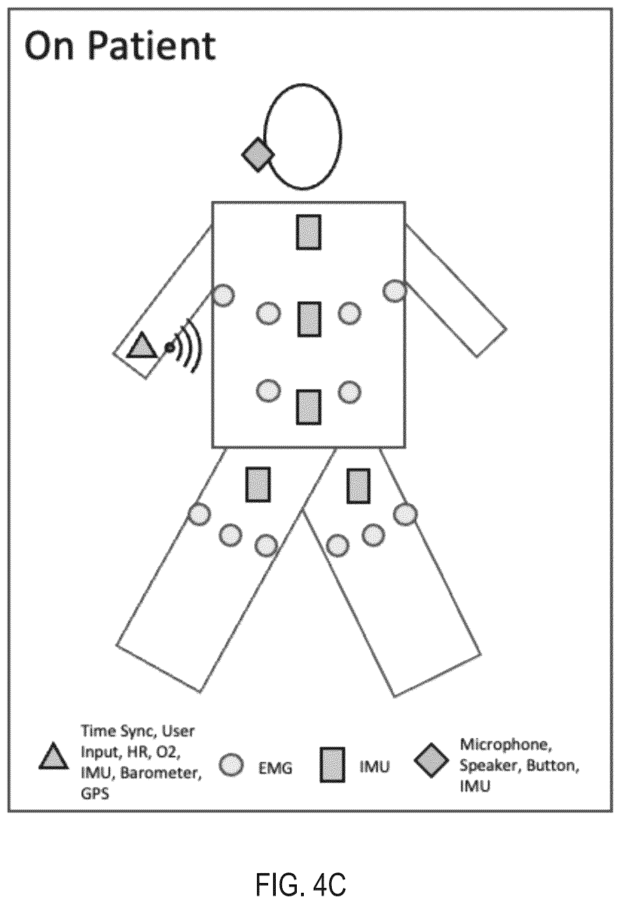

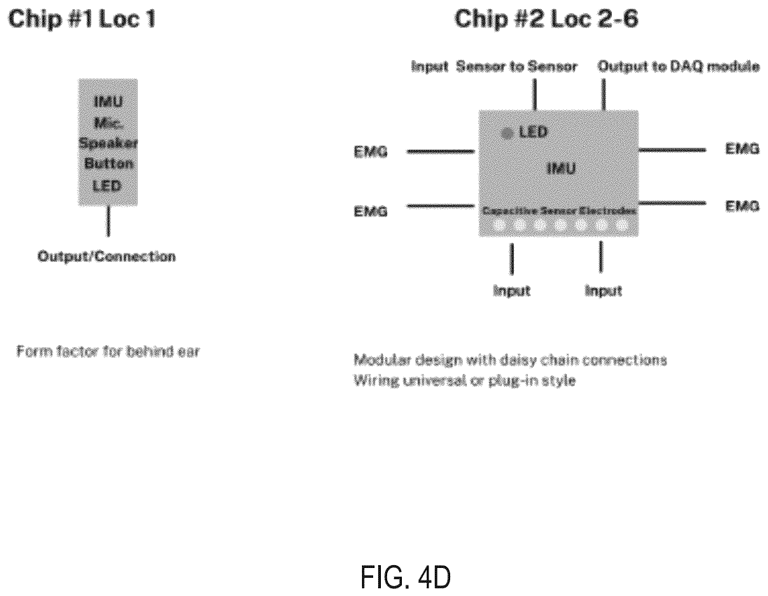

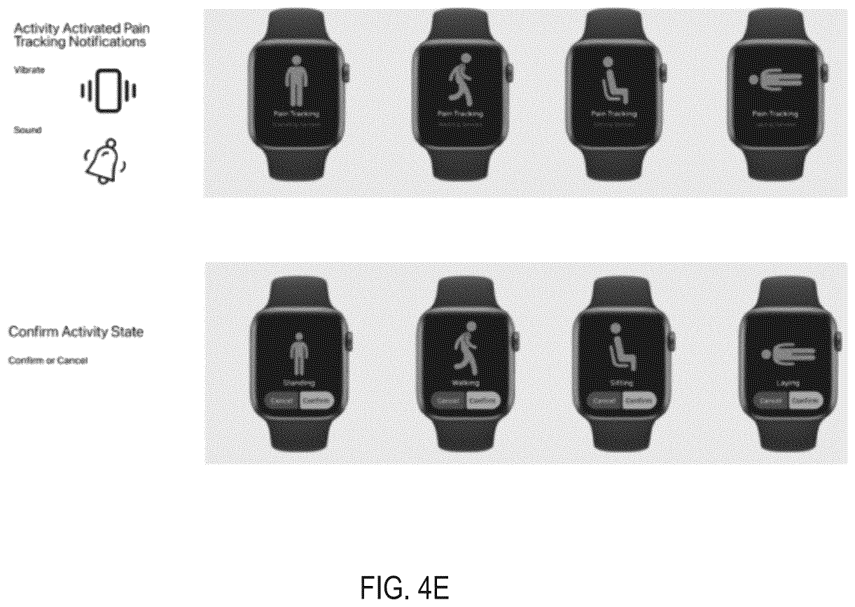

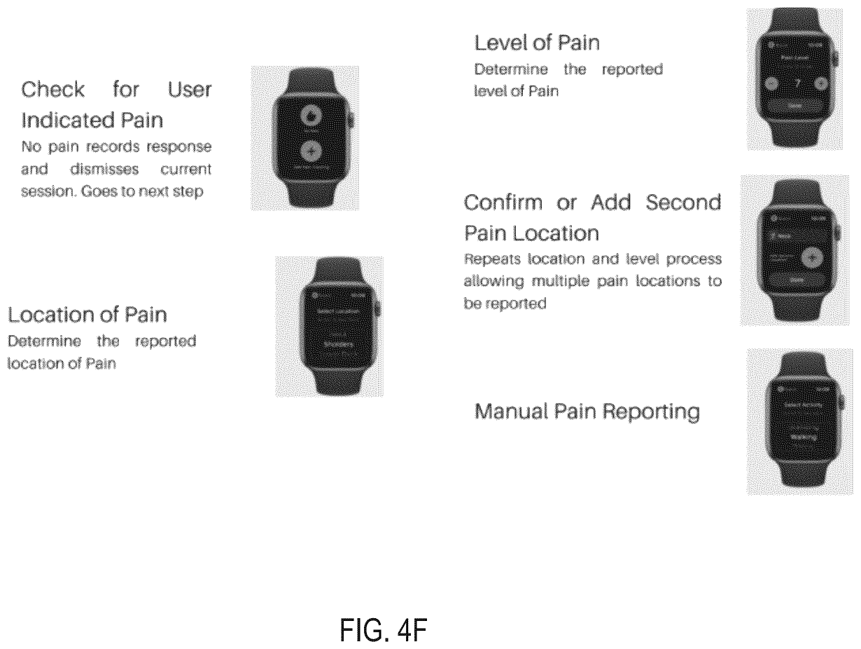

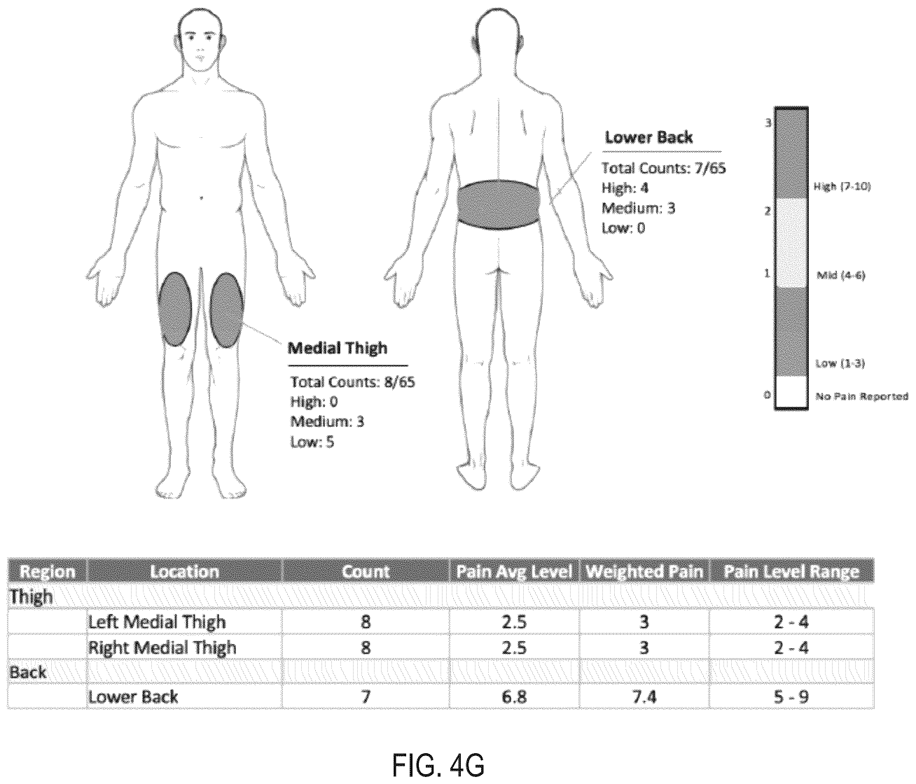

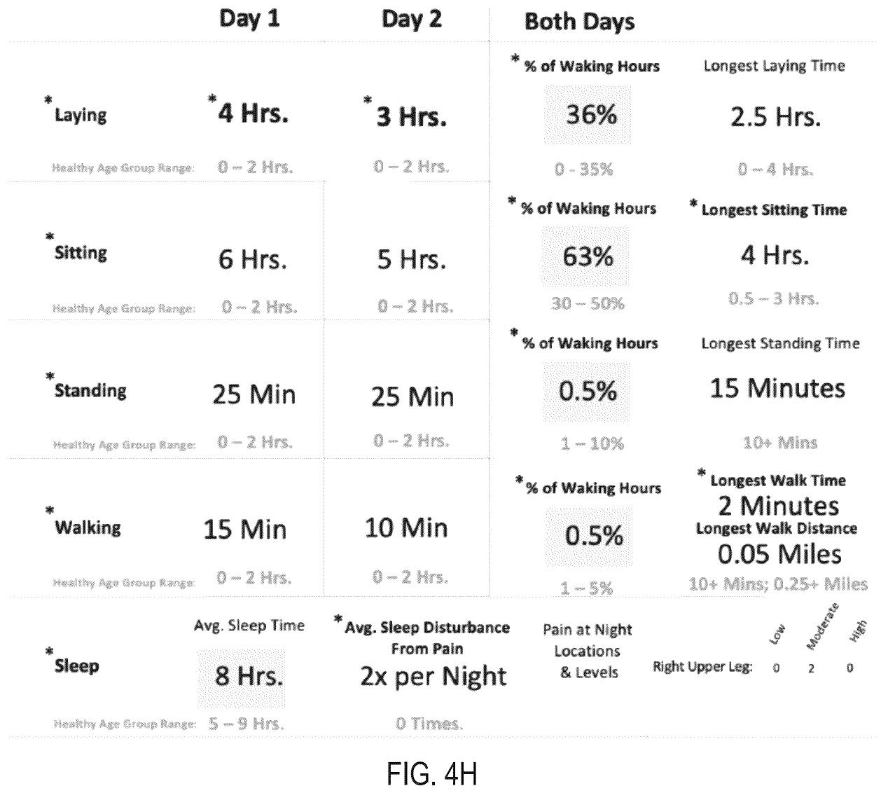

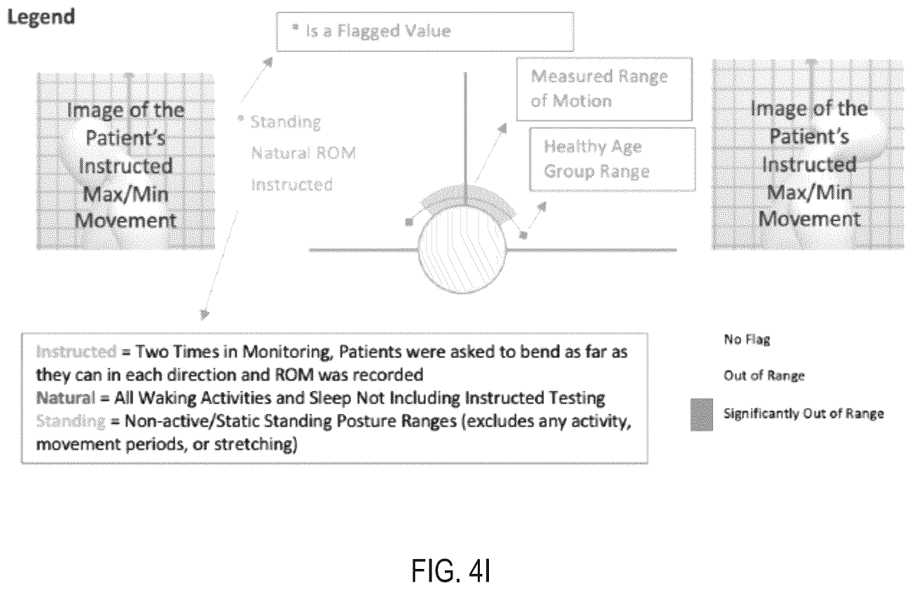

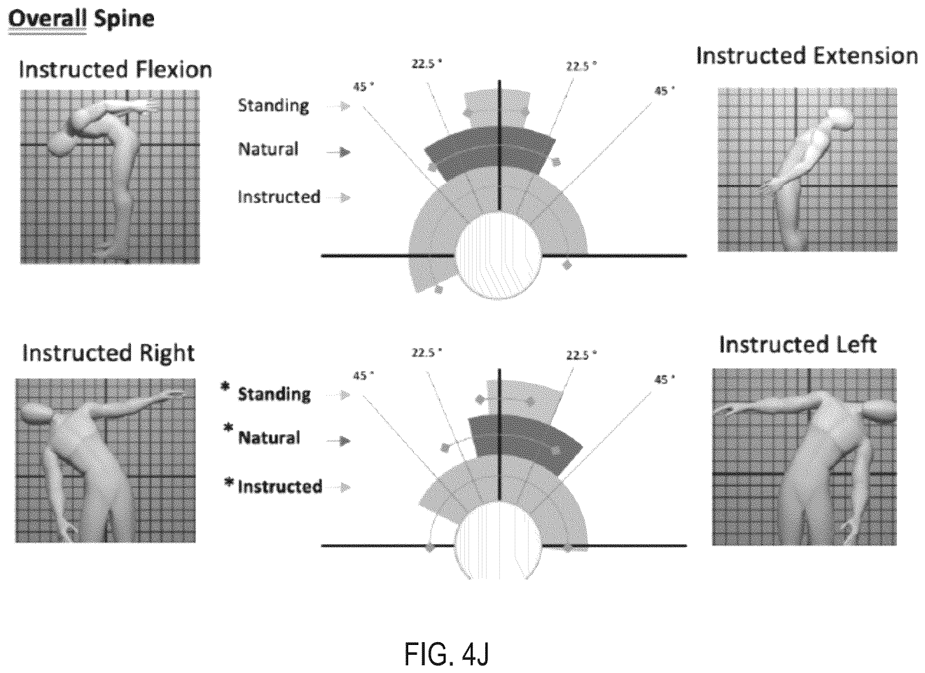

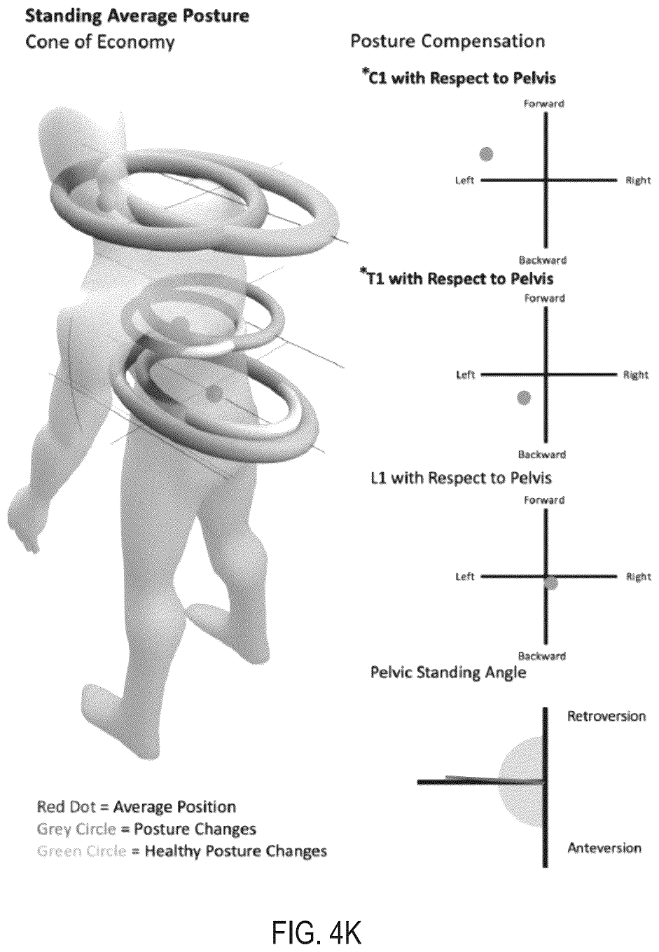

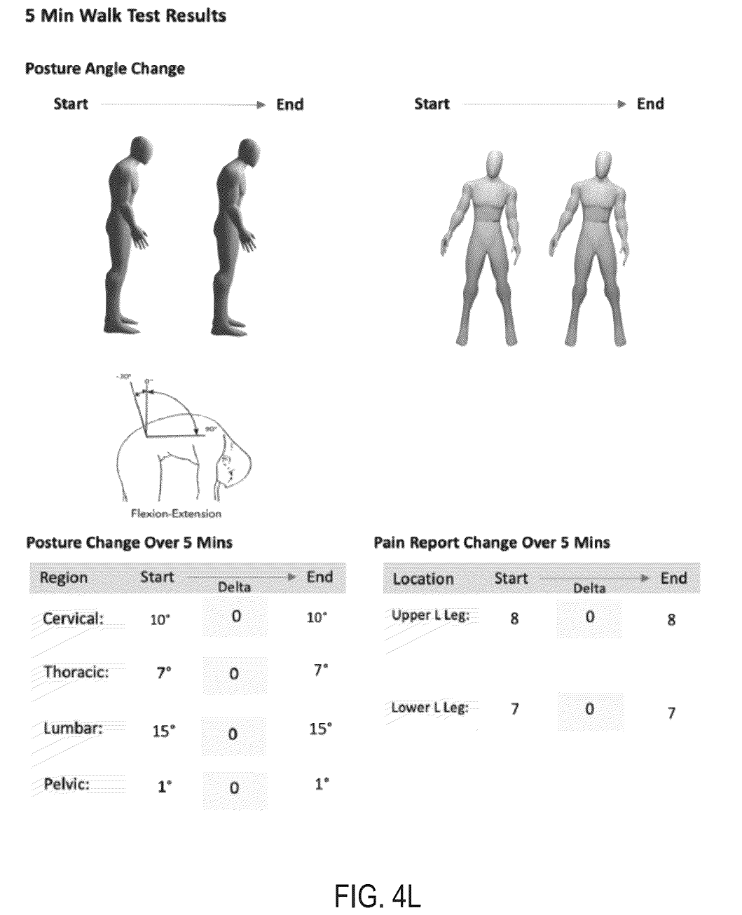

According to some embodiments, as discussed in more detail below, assessment engine 200 may be configured to implement and/or control a plurality of services and/or microservices, where each of the plurality of services/microservices are configured to execute a plurality of workflows associated with performing the disclosed patient monitoring and management. Non-limiting embodiments of such workflows are provided below in relation to at least FIGS. 4A-4L, and the included disclosures in APPENDIX A as accompanying the filing of U.S. Provisional Application No. 63/442,984, which is incorporated herein by reference in its entirety, as discussed supra.

According to some embodiments, as discussed above, assessment engine 200 may function as an application provided by cloud system 106. In some embodiments, engine 200 may function as an application installed on a server(s), network location and/or other type of network resource associated with system 106. In some embodiments, assessment engine 200 may function as an application operating via a conventional edge device (not shown) at location associated with system 100. In some embodiments, engine 200 may function as application installed and/or executing on UE 102. In some embodiments, such application may be a web-based application accessed by UE 102, peripheral device 110 and/or devices associated with sensors 112 over network 104 from cloud system 106. In some embodiments, engine 200 may be configured and/or installed as an augmenting script, program or application (e.g., a plug-in or extension) to another application or program provided by cloud system 106 and/or executing on UE 102, peripheral device 110 and/or sensors 112.

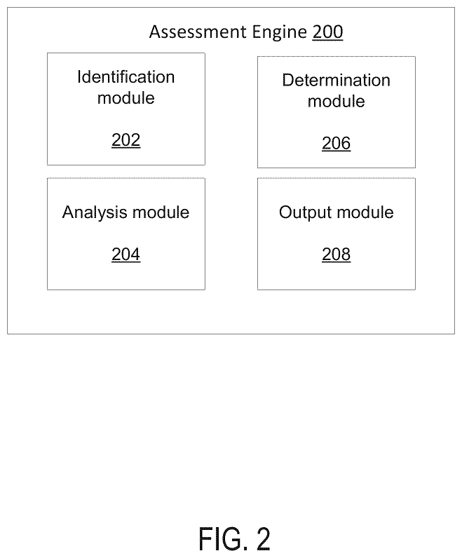

As illustrated in FIG. 2, according to some embodiments, assessment engine 200 includes identification module 202, analysis module 204, determination module 206 and output module 208. It should be understood that the engine(s) and modules discussed herein are non-exhaustive, as additional or fewer engines and/or modules (or sub-modules) may be applicable to the embodiments of the systems and methods discussed. More detail of the operations, configurations and functionalities of engine 200 and each of its modules, and their role within embodiments of the present disclosure will be discussed below.

Turning to FIG. 4A, Process 400 provides non-limiting example embodiments for the disclosed framework. According to some embodiments, Process 400 provides computerized mechanisms for the disclosed dynamic spinal assessment discussed herein.

By way of background, ASD, in particular, is a common disorder that causes significant quality-of-life burdens, affecting approximately 27.5 million elderly patients. This number will continue to grow as the population ages. Treatments for ASD are currently expensive, ranging from $10,8153 to $87,0004 for non-surgical and surgical interventions, respectively. The primary surgery for ASD is a spinal fusion, yet nearly 20% of these procedures pose a significant risk of complications and have inadequate patient outcomes. However, rates of these procedures are growing. Between 1998 and 2008, cervical, thoracic, and lumbar fusions rose by 90%, 61%, and 141%, respectively. 17% of spine surgeries performed were determined to be on patients who should not have been recommended surgeries. Worse yet, many spinal fusion patients face revision surgeries in the first few years due to failed back surgery syndrome. For example, lumbar fusion procedures have a revision rate of up to 36%. Patients navigating this uncertain treatment pathway often feel hopeless and are not receiving the treatment they need due to a lack of patient-specific metrics to guide treatment.

The spine is a mobile structure that allows the body to bend, twist, and lift. However, spine surgeons currently have no method of quantitative motion analysis in their current diagnosis and surgical planning for ASD patients. Current treatments are based on static images such as X-Rays, CT scans, and MRIs in conjunction with short clinical visits. A significant component of surgical decision-making has long been based on clinical observations. However, clinical visits only provided qualitative information for the surgeons to use. Surgeons would ask the patient about their motion, posture, and pain at home and throughout the day, but no quantitative method to characterize a patient's spine is currently available.

Thus, there currently exists a critical unmet need in providing physicians with the ability to dynamically assess spinal patients. As provided herein, the disclosed systems and methods can provide a set of key dynamic metrics that identify, address and are associated with the root cause of existing inadequacies and inconsistencies in spinal patient treatment planning. For example, such dynamic metrics include, pain, body position, muscle activity, activity level and biological parameters.

According to some embodiments, “pain” can correspond to, but not be limited to, values, metrics and/or other forms of data/metadata indicating the causes of pain, trends in the time or severity of pain, location on the body, frequency of pain, and the like, or some combination thereof.

In some embodiments, “body position” can correspond to, but not be limited to, values, metrics and/or other forms of data/metadata indicating posture, spinal motion, hip, knee, and/or leg positions, flexibility, gait measurements, the posture of different activities, pain-producing postures, posture changes with fatigue, spinal or posture compensations (pelvic tilt, leg position, muscle usage, and the like), and the like, or some combination thereof.

In some embodiments, “muscle activity” can correspond to, but not be limited to, values, metrics and/or other forms of data/metadata indicating surface EMG data, needle EMG data, muscle fatigue, neural activity, radiomyography (RMG), ultrasound, magnetic resonance elastography (MRE), and the like, or some combination thereof. In some embodiments, “muscle activity” may be indirectly measured through the use of accelerometers, gyroscopes, or magnetometers. In these situations, muscle twitches, changes in motion, and changes in vibrations may be utilized to approximate muscle activity indirectly. These activities may be approximated and may be calculated with a model between acceleration and vibrations related to material or skin tension or changes in material properties. In addition, localized motions with respect to global motion may be analyzed to find features of the sensor motion related to muscle activity.

In some embodiments, “activity level” can correspond to, but not be limited to, values, metrics and/or other forms of data/metadata indicating steps, standing time, walking time, distance, fatigue throughout the day, and the like, or some combination thereof.

In some embodiments, “biological parameters” can correspond to, but not be limited to, values, metrics and/or other forms of data/metadata indicating HR, 02, Respiration, and the like, or some combination thereof.

Accordingly, these metrics can influence, for example, “If the patient should receive surgery,” “How the patient will respond to treatment,” “What type of surgery is optimal,” “What levels and areas to focus on for treatment,” “If the patient is at high risk for future nerve damage,” “The risk profile of the patient,” and/or “What is their optimal non-operative treatment path,” “What implant to use?”, What correction angle is optimal?”, “When to do surgery?”, “What treatments would be effective?”, and the like.

According to some embodiments, the disclosed systems and methods can provide computational DI-based mechanisms for addressing issues within non-operative and operative ASD treatment. It should be noted, however, as discussed above, that while the discussion herein is focused on ASD, it should not be construed as limiting, as other symptoms, conditions and/or statuses of patients, inclusive of healthcare and non-healthcare environments, can be addressed via the disclosed systems and methods without departing from the scope of the instant disclosure.

In some embodiments, the disclosed framework can inform the patient treatment pathway with quantitative metrics to guide faster, more effective, and targeted care. In some embodiments, the disclosed framework can provide patient-specific dynamic factors that surgeons currently lack in their surgical planning, which can lead to significant post-operative complications. In some embodiments, the disclosed technology provides a clear and significant value to the full chain of stakeholders in the spine care market by reducing ineffective treatments, improving surgical outcomes, and improving patient communication.

According to some embodiments, as evident from the instant disclosure, the disclosed framework can have and/or involve revenue-generating sources. For example, the framework can operate via a per-patient prescription test fee. In some embodiments, the framework can involve a follow-up assessment that will be done after the course of treatment to evaluate the patient's changed status. In some embodiments, another revenue-generating source can be associated with the brokerage of the collected data to medical device companies (and/or other forms of third party entities). As such, in some embodiments, the disclosed framework has significant value in providing pre-operative (and intra- and post-operative) insight into patient-specific data to enable improved treatment. These insights are applicable and necessary for the future of spinal care—from inter-operative guidance to custom implants to custom robotic procedures and fit into the current shift toward value-based care metrics.

According to some embodiments, as discussed in more detail below, the disclosed framework can operate via captured metrics about the wearer of the device/sensor. In some embodiments, such metrics may be dynamic or static and can include, but are not limited to, posture, pain, motion, activity, muscle activation, and the like. According to some embodiments, the device/sensor can be placed on a patient, after which the disclosed framework can be calibrated to the wearer, often through a set of movements. The wearer then wears the system which enables the disclosed framework to collect data throughout the time the device/sensor is worn. The system can then be removed (e.g., which may be optional), whereby the collected data can be leveraged for the generation of the clinical report about the patient.

In some embodiments, the generated report can have relevant to, but not limited to, medicine, exercise, training, ergonomics, sports, rehabilitation, and the like, or some combination thereof.

According to some embodiments, Steps 402, 406 and 408-414 of Process 400 can be performed by identification module 202 of assessment engine 200; Steps 404 and 416 can be performed by analysis module 204 and/or determination module 206; and Step 418 can be performed by output module 208.

In some embodiments, as provided below, Process 400 can involve Steps 402-418, which as provided below, respectively involve placement, calibration, sensing, user instruction, live monitoring, removal, upload, analysis and data review. In some embodiments, the steps provided in Process 400 related to user instruction (Step 408), live monitoring (Step 410), removal (Step 412) and upload (Step 414) may be optional, and/or performed in a different order as depicted in FIG. 4A.

According to some embodiments, Process 400 begins with Step 402 where the disclosed UE (e.g., UE 102, for example; or sensor 112, as discussed above) is placed on and/or near the subject. For example, a sensor array, as discussed herein. In some embodiments, Step 402's placement can involve components that include sensors that are used to collect data on the wearer. In some embodiments, the components can be placed individually on the skin of the wearer, or they may be embedded into a garment. In some embodiments, some sensors may require adhesion to the skin.

In some embodiments, Step 402 may involve shaving of the area for removal of hair, abrasion of the skin surface, cleaning the surface with soap and water or wiping with alcohol to clean the surface, and application of the adhesive material to the skin. In some embodiments, a mark may be used to designate the location of the device on the wearer for placement. In some embodiments, such mark may serve as a future reference to be used in the calibration system. In some embodiments, such mark may also be used in case of the sensor removal or the sensor falling off to realign and place the sensor. In some embodiments, the device, adhesive, garment, template, and/or tool may mark on the skin to note the location. In some non-limiting examples, the adhesives could be lined with ink or other agents for marking of location on the wearer.

In some embodiments, if the components include a garment, the garment can be, but is not limited to, a shirt, vest, unitard, or any other shaped fabric. In some embodiments, the garment may also be a template or tool that is used to help locate the placement of the system components on the subject. In some embodiments, the components may be left on for the duration of the sensing or removed after the placement of the sensors. In some embodiments, the garment may be customized or fitted to the wearer. In some embodiments, the garment may also contain markings on it to help with the orientation and instruction of placement for the user. In some embodiments, the markings may also be used to aid in the segmentation of the frames captured in calibration steps.

In some embodiments, the components may be incorporated into a garment to aid in the placement and accuracy of the tracking system. In some embodiments, the garment may be a compression shirt with additional fabric on the legs (or other portions of the body). In some embodiments, the components have spots where they insert into the garment leaving open areas for the intended location of the component to be affixed to the garment. Through these holes in the garment to indicate the location of the sensors, the skin can be shaved and prepped for adhesion.

In some embodiments, such sensors can be turned on and the film covering the adhesive can be removed. In some embodiments, the sensors can then adhered to the skin and clipped into the garment.

In some embodiments, instructions for the placement occurring in Step 402 may originate from a digital device, such as a tablet, for example. In some embodiments, the screen or interface of the tablet may show instructions depicting how to best place the sensors. In some embodiments, the screen or interface may show graphics, animations, pictures, and or any other media to convey the instructions. Additionally, in some embodiments, instructions may be written, visual, auditory, and/or provided haptically.

In some embodiments, a computer system, for example, a tablet, and a camera may give real-time feedback on the placement of the sensors. In some embodiments, a camera may aid in the placement of the sensors. The camera may be embedded in a computer system, such as a tablet. In some embodiments, images (or frames) from the camera may serve to augment the physical scene, overlaying or changing components of the visual scene to provide instructions to the user. In some embodiments, images from the camera may also be analyzed by the computer system to detect objects in the image. In some embodiments, tracked objects in the image may include the person, the garment, the sensor(s), and/or anatomical landmarks. In some embodiments, computer vision and object detection may be used for these tracked objects in order to provide feedback to the user for the placement of the sensors, as discussed below. In some embodiments, engine 200 may output and provide guidance to the user as to the location and optimal placement of the sensor. In some embodiments, it may also be used to validate or check the placement of the sensor or garment.

In some embodiments, an imaging device, for example, a CT, MRI, or X-ray, can be utilized to determine an optimal location on the wearer (e.g., based on their underlying anatomy). Thus, for example, a CT image of a patient can be captured, and engine 200 can analyze and segment the image to determine the markers for which placement of the components in Step 402 can be performed. In some embodiments, one or more images could be used to calculate postures or bone positions with respect to known body positions in order to give more precise measures of body position within the system. In some embodiments, these images may be used in order to measure wearer measures such as skin thickness for sensor offset calculations. It may also be used to calculate centers of rotation for use in sensor processing. In some embodiments, this data may be used to predict the motion or loading of bones, muscles, or ligaments from the sensor systems. The modeled relationship between the sensor system and the underlying anatomy may be used in this prediction. In some embodiments, these images may be used to calculate bone properties such as bone density, volume, or other parameters that may be used in the analysis of the data. In some embodiments, these images may be used to recreate or model the wearer's anatomy in order to more accurately model and predict force, alignment, and/or motions. These models may be used in the planning and modeling of possible interventions for the patient. These models may be simulated using FEA or any known simulation method. Multiple inputs can also be used in the model, such as the surgical intervention to be used or the surgical hardware to be used. In some embodiments, the dynamic measurements from the sensor system may be used in the modeling of the anatomy in order to provide more accurate loading scenarios. In certain embodiments, data from other surgeries may be fed into the model in order to better predict outcomes.

In some embodiments, as mentioned above, such analysis and segmentation can be performed by engine 200 utilizing any type of known or to be known AI/ML algorithm or technique including, but not limited to, computer vision, classifier, feature vector analysis, decision trees, boosting, support-vector machines, neural networks (e.g., convolutional neural network (CNN), vision transformers (ViTs), recurrent neural network (RNN), and the like), nearest neighbor algorithms, Naive Bayes, bagging, random forests, logistic regression, and the like.

In some embodiments and, optionally, in combination of any embodiment described above or below, a neural network technique may be one of, without limitation, feedforward neural network, radial basis function network, recurrent neural network, convolutional network (e.g., U-net) or other suitable network. In some embodiments and, optionally, in combination of any embodiment described above or below, an implementation of Neural Network may be executed as follows:
a. define Neural Network architecture/model,
b. transfer the input data to the neural network model,
c. train the model incrementally,
d. determine the accuracy for a specific number of timesteps,
e. apply the trained model to process the newly-received input data,
f. optionally, and in parallel, continue to train the trained model with a predetermined periodicity.

In some embodiments and, optionally, in combination of any embodiment described above or below, the trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the trained neural network model may also be specified to include other parameters, including but not limited to, bias values/functions and/or aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the aggregation function may be a mathematical function that combines (e.g., sum, product, and the like) input signals to the node. In some embodiments and, optionally, in combination of any embodiment described above or below, an output of the aggregation function may be used as input to the activation function. In some embodiments and, optionally, in combination of any embodiment described above or below, the bias may be a constant value or function that may be used by the aggregation function and/or the activation function to make the node more or less likely to be activated.

In some embodiments, engine 200 can utilized a pose estimation algorithm which can be utilized to calibrate the movements and/or poses captured during processing of Step 404.

According to some embodiments, for example, CT or X-rays can aid engine 200 with determining the optimal location to place the devices on the wearer based on their underlying anatomy. In some embodiments, information from imaging systems such as CT, ultrasound, MRI, X-ray, or any other imaging modality may be utilized to determine parameters for sensor placement, garment fit, placement guidance, or any other activity pertaining to the calibration or location of the sensors. In some embodiments, key anatomical landmarks may be identified in the images and measurements or relationships may be determined. In some embodiments, relationships may be distances, curvature, angle, size, shape, circumference, location, orientation, or any other measurable parameter of a landmark or multiple landmarks.

In some embodiments, the sensors may be worn at the same time as images, for example, CT, X-rays, MRI. In some embodiments, the timestamp or measurement period of the system may be related to the time of image. In some embodiments, simultaneous images and sensor readings may be used to aid in the calibration of the system. In some embodiments, this approach may provide extra information about the images themselves to the provider, such as the body position with respect to the overall range of motion and/or the motion of the individual section of the tracked body parts captured, or not captured in the images. In some embodiments, this approach may be used to determine compensation motion by the wearer to accommodate imaging positions.

In some embodiments, measures such as patient height, leg length, joint angles or positions, spinal segment angles, vertebral body angles, and bone lengths or angles may be determined in this method and utilized by engine 200. In some embodiments, these relationships may be used to determine ideal placements of the sensor(s) or provide information in the analysis of the data. In some embodiments, these sensor placements would be optimized to track the full range of motion of the desired body parts. In some embodiments, they may also be used to determine the location of the starting and ending sections of joints. In some embodiments, these measurements may be used to create templates to more easily placed sensors. In some embodiments, these measurements may also be used to guide the instruction of sensor placement. In some embodiments, these measures may also be used to customize the garment or holder of the sensors to the patient.

In some embodiments, palpation of the skin may be utilized in order to determine the proper location for sensors to be placed. This may be in conjunction with other embodiments, as discussed herein.

According to some non-limiting embodiments, for example, ultrasound may be utilized in order to locate points for placement. In some embodiments, ultrasound may be guided by a user and key landmarks determined for the placement of the sensor. In some embodiments, signals from the ultrasound may be analyzed and used to find the correct location or position for the sensor. In some embodiments, this guidance may use artificial intelligence in order to determine the proper location for sensor position and orientation.

According to some embodiments, some sensors can be co-located or affixed/placed as combinations on a portion of the patient, and in some embodiments, some sensors may be configured and used for specific locations on the patient (e.g., specific body parts, for example). For example, EMGs can be placed on specific muscles.

In some embodiments, sensor combinations can be augmented to cover different parts of the body including the lower back, the mid-back, the neck, behind or anywhere near the ear, the torso, the chest, the hip, the pelvis, the knee, the elbow, the wrist, the ankle, one or more of the fingers, and other extremities. In some embodiments, sensors can additionally contain EMG and/or other muscle activity measurement sensors described herein. In some embodiments, sensors may be placed over areas with muscular activity that needs to be monitored or stimulated for a number of reasons including longitudinal disease tracking, fatigue measurements, neurostimulation training, detecting muscular tightness, muscular health, training, activation during activity, muscular death, etc. In some embodiments, the EMG-based sensors may make up all of the sensors on the body, some of the sensors on the body, or none of the sensors on the body.

In some embodiments, some sensors may need to be placed on certain parts of the body due to the shape of the sensor, accommodation of fat rolls, the function of the sensor (voice activation for example), influence on calibration, etc. In some embodiments, some sensors may not be specific to the body and may be flexible on their location.

In some embodiments, the sensors can be applied and/or affixed to the correct spots via a customized approach, which can be based on a shape of the interface of the sensor and/or numbers on the device. In some embodiments, an extra adhesive patch may be used to go over the top of a sensor to keep it in place on active patients.

In some embodiments, additional material or adhesives may be utilized in order to further secure the sensor(s) to the patient or garment. This may be a patch or strap that goes over the top, around, attaches to, or any other method of providing support to the sensor(s). These additional materials may have adhesive, hook and loop, and/or any other method to attach to the user, garment, or object the sensors are attached to.

In some embodiments, the sensors can be centered and placed on a patient according to a customized approach, as disclosed herein. In some embodiments, this can involve the addition of a small plastic panel that can be laser-cut for specific patient dimensions. Once the sensor is placed in the center of the plastic panel, the user lifts up the panel which may simultaneously removes the adhesive on the back of the sensor and helps depress the sensor in the correct location. In some embodiments, such approach can be incorporated with a compression shirts or compression suit that can be worn by a patient (e.g., a garment). In some embodiments, the medical condition of the patient may be used to inform the optimal sensor placement(s).

In some embodiments, the placement of the sensors may be aided by the inclusion of additional components. In some embodiments, such additional components may help with the centering, placement, and or location of the sensor. In some embodiments, such components may be sized in order to adapt to the wearer of the device. In some embodiments, the components may be manufactured in different sizing options or custom-made for the wearer. In some embodiments, the components may be made of plastic or other materials. In some embodiments, the components may contain features such as supports that can attach to the garment. In some embodiments, they may also aid in protecting the adhesive of a sensor from other objects until it is time for the sensor to be placed. In some embodiments, they may remain on the wearer or garment, or they may be removed.

By way of a non-limiting example, in some embodiments, the components of the sensors (and/or the sensors as a whole), may be a laser-cut piece of plastic that folds onto itself and attaches to the sensor. In some embodiments, there may be tabs that attach to the garment. In some embodiments, such tabs may vary in length depending on the wearer's size in order to keep the sensor in position at the optimum location. In some embodiments, after preparation of the skin, the user may pull the top of the plastic piece which unfolds the plastic exposing the adhesives of the sensor while still maintaining support to keep the sensor in the proper location, after adhering the sensor, this may be removed from the garment.

In some embodiments, the placement of the sensors may be aided by the use of a template. In some embodiments, the template may be localized to a known anatomical location. In some embodiments, the template may include holes and/or marks that aid in the placement, location, and or orientation of the sensors. For example, the template may be a plastic template with holes throughout the center of known spacing accordingly for optimum sensor placement. In some embodiments, the sensors may be placed through the hole and affixed to the wearer. In some embodiments, after the sensors are affixed, the template may be removed.

In some embodiments, Step 402 can involve activation and assignment of an identifier (ID) for the device/sensor and/or patient. In some embodiments, this information can be stored in database 108, which can be stored along with biographic and/or demographic information about the patient, inter alia other forms of data related to the patient and/or procedure.

In some embodiments, information may be provided in the set-up of the device. In some embodiments, the information may be input into the system through a computer interface by the wearer or another individual. In some embodiments, may aid in the process identifying the wearer and linking the data to that individual. In some embodiments, additional information may be provided such as age, weight, birth date, patient ID number, doctor, address, or any other biographical information useful in linking the device. In some embodiments, such data may be used later to retrieve information from the system. For example, such information may be provided by a prescribing physician entering information about a patient in an online portal in order to prescribe the system. In some embodiments, the doctor may enter information into a graphical user interface (GUI) in order to register the patient to a device. In some embodiments, the same information may be utilized in the retrieval of the data from the system at a later point. In some embodiments, another GUI may be utilized in order to access the information from the sensors using the information that the doctor or technician has provided for the prescription of the device.

In some embodiments, engine 200 may receive data about the wearer from a third party, such as but not limited to, electronic medical records or fitness plan applications. This data may be stored by the system and/or used in the processing of the data. In some embodiments, this may be used in conjunction with the manual entry of data.

In some embodiments, engine 200 may send data to a third-party system, such as but not limited to, electronic medical record or fitness plan application. In some embodiments, such data may be used to visualize data from the system and/or store data from the system.

In some embodiments, users may input certain goals or targets for the wearer. In some embodiments, such goal/target information may come in the form of certain activity goals for the wearer to achieve. In some embodiments, it also may come in the form of activities or motions that are desired for the individual. In some embodiments, this may be a regiment such as stretching or other forms of exercise. In some embodiments, they may also be any activities or limitations on wearer, such as range of motion limitation, exercise limitation, heart rate limitations or any other metric tracked by the system. In some embodiments, such goals or metrics may be tracked against the actual execution of the tasks by the wearer. In some embodiments, they may also be transmitted back to an electronic system to track the success of the tasks.

For example, in some embodiments, an inputting user may be a personal trainer setting the goals for a patient going through physical therapy after a spine surgery. In some embodiments, they may request that a patient stretches every day through a set of different motions or movements. In some embodiments, such information may be relayed back to the user, or to the individual who set the goals.

In another non-limiting example, the information may come in the form of restrictions after a surgery or an injury, where such restrictions may be a certain amount of time standing or certain movements that could risk the patient. In some embodiments, after a spine surgery, a doctor may input into the system that the user must not exceed 30 degrees of cervical flexion. In some embodiments, engine 200 can track these requirements and even provide feedback to the user. In some embodiments, the user may get alert on their system of a potential breach (or impending breach based on one or more trends) of set parameters. In some embodiments, such an alert may come in the form of vibration, sound, push notification, and or visual display.

In Step 404, engine 200 can effectuate calibration of the placed device (from Step 402). In some embodiments, after the sensors have been affixed to the subject (in Step 402), calibration can take place. In some embodiments, calibration can be based on a set of particular movements or poses. In some embodiments, such poses can include, but are not limited to, bending forward, looking in different directions, twisting, walking, and crouching down, and the like. In some embodiments, the wearer may sway or rotate tracked body parts in particular anatomical planes in order to calibrate the system.

In some embodiments, the posture and/or body position on the wearer may be analyzed in relation to physiological parameters such as heart rate, blood pressure, respiration rate, temperature, oxygen saturation, glucose levels, blood oxygen saturation, cerebral blood flow, electrical activity of the brain and or any other physiological signals captured by the system. This may be used in the diagnosis, prevention, and/or treatment of conditions such as vertigo, instability, and falls, Postural Orthostatic Tachycardia Syndrome (POTS), or any other condition where the relation of physiological measures in relation to motion and activity is relevant. According to some embodiments, by way of a non-limiting example, a patient experiencing dizziness and fatigue may present to the clinic. A doctor may prescribe the use of our wearable system to the patient. The system may include one or more photoplethysmography (PPG) sensors to track heart rate and blood oxygen levels. The system may record and time sync the motion data and the data from the PPG sensor to detect the changes in heart rate as associated with POTS upon standing. The algorithms may look for trends over time and use the motion characteristics to further validate the symptoms.

In some embodiments, calibration can involve utilizing a camera system, phone, or tablet, whereby the motions can be recorded. In some embodiments, during the calibration period, the worn sensors record measurements while the video is taken. Using the video taken of the subject, these images can be analyzed using AI/ML (e.g., computer vision) to segment the subject and estimate the pose of the subject. In some embodiments, the pose and joints of the subject can be extracted using AI/ML to segment and assess the position of points of interest on the subject and the system. In some embodiments, measurements taken from video analysis can be used to calibrate the sensors affixed to the subject. In some embodiments, after calibration, the disclosed framework can run independently to capture the desired motion and physiological metrics without the need for external camera systems.

In some embodiments, a GUI and/or UE may be used to aid in the connection of devices. In some embodiments, the user may connect a wireless camera, smartphone, and/or tablet to the system with a QR code, NFC connection, RFID, or the like to the system. This may serve as an external sensor for the system. In some embodiments, a smartphone may act as an external device by scanning a QR code with a deep link in order to associate the external device with the patient record. This device may be used in order to record video of the wearer of the system. In some embodiments, the external device may be used to link the sensors to the patient record through QR code, NFC, RFID, or the like. The sensors or the housing of the sensors may contain the QR code, NFC, RFID, or the like that can be used to communicate information about the sensors such as serial number, status, battery, storage, device time, or any other data from the system. The system can also send data to the sensors via the same mechanisms to write data to the sensor system.

In some embodiments, an external device to add data to the system may be used with or without the sensors. The data may be used to inform the system or provide data back to the users. In certain embodiments, a mobile phone may be used to record the video of the wearer, with or without the sensors. The wearer may be instructed to walk, stand still, perform physical exercise, stretch, bend forward, bend backward, twist, squat, lead, bend their head, lay down, or the like. The video may be analyzed to capture information such as gait measurements including but not limited to, stride time, stride length, stride symmetry, two stance phase time, directional deviation, shoulder tilt, pelvic tilt, or metrics such as, but not limited to, range of motion, posture, spinal alignment, or spinal curvature. These metrics may be calculated using any combination of the methods disclosed in this application. These metrics may be tied back to the wearer's data in the system or future data collected by the system, such as the worn devices.

In some embodiments, measurements using external devices may be taken at other times than the calibration period. In some embodiments, these measurements may be prompted by a notification, text, alarm, email, or phone call, either automated or manually instigated. These measures may also be done by the wearer at prescribed times, upon changes in symptoms, or any other reason. These measurements may be, but not limited to, a video, photo, audio recording, weight measurement, heart rate measurement, blood oxygen measurement, rapid diagnostic test, or glucose reading. According to some embodiments, by way of a non-limiting example, the system may be used without the worn sensors to monitor a physical therapy patient getting treatment for a knee joint replacement. The doctor may program into the system a text notification requesting measurement of both range of motion video and gait video for the patient every three days. The patient may be notified via text and click a provided link to access the instructions and recording software on their mobile phone. The software may monitor the motion or even count down time to start the prescribed measurements. The software then may perform some analysis live on the mobile application, such as blurring the face of the patient and correcting for light exposure. The data may be transmitted via cellular connection or WIFI to a storage server in the cloud. The action may trigger a remote computer to run further analysis using a AI/ML model for pose estimation and image detection. From these detected points, other models may be used to smooth the data and/or fill in the gaps of the data. Other models may be included in order to intuit 3D data or meshes from the scene. Other models may be used to calculate the location of scene objects, such as the floor and used to transform the collected data or to detect events such as steps. The data may be processed and reports generated such as joint range of motion, joint stability, walking speed, varus or valgus knee angle measurements, or any other measure of interest dictated by the care provider. This data may be logged in a database to be viewed by both the patient and the provider. The trends over time may be calculated after multiple sessions and progress may be mapped to predicted outcomes. The disclosed algorithm may be used to flag potential risk factors to the patient to inform the provider for future intervention. This data may also be used by the physical therapist to proscribe new exercises to address these flagged risk factors.

According to some embodiments, by way of a non-limiting example, a patient with Parkinson's or Multiple System Atrophy (MSA) may be notified on their smartwatch to take a recording of their voice. The watch app may give a prompted script for the patient to read. The voice of the patient may be recorded and uploaded to the cloud for analysis. Feature of voice detection, such as, but not limited to, cadence, inflection, spectrum flatness and spectral distribution of energy, hoarseness, articulation, phonation, prosody, vocal intensity, loudness variability, fundamental frequency variations, speech rate, breath support, fluency, dysarthria, aphasia, jitter and shimmer may be determined. An overall score may be calculated representing the accumulation of multiple factors to provide to the provider as well as a full analysis of the features. This data may also be fed into the future uses of the worn sensor system as a comprehensive report of Parkinson's progression. This data may be tracked over time by the provider and trends throughout the day or over the course of the week may aid in the dosing and prescription of medication or selection of treatment.

According to some embodiments, by way of a non-limiting example, a doctor treating a patient recovering from a stroke may place the wearable sensors on the arms and legs of the patient for a two-week period after the stroke to follow the progress of the patient. The doctor may schedule activity to be performed once a day activity and the system may remined to the patient via email. The patient may receive an email with the day's activities. The link on the email brings them to a page containing a form of questions or inputs, for example the SF-36 health survey and the Lawton Instrumental Activities of Daily Living Scale. After the survey is complete the interface may prompt the patient to perform a repetitive motor training task such as finger tracking. The system may access the user's camera and in addition to the worn sensors to track the fingers and the muscle activity signals of the patient performing the task. The system may use computer vision to track the motions of the patient and provide feedback for the session. Data from the session may be tracked to track functional status changes. The user may see an animated gamified version of the finger tracking exercise that shows them moving balls into virtual baskets on the screen. This data may be captured remotely and analyzed on the patient's computer system locally and only the data from the outputs of the tracking may be uploaded to the servers in the cloud to prevent the transmission of videos with identifying features being stored on the system. The doctor may receive a push notification telling them that all of the patients that day except for one completed their daily task. The doctor may have the ability to reach out via a phone call to the patient who missed the exercise of the day to check in on their progress.

According to some embodiments, by way of a non-limiting example, a clinic that treats spine patients may use the system as a pre-assessment, during evaluation assessment, or post-assessment. A patient with lumbar degeneration and cervical myelopathy may make an appointment with a doctor's office. The doctor's office may input the basic patient information into the GUI of the system. The system may send a text message reminder to the patient to come to the clinic. When they arrive at the clinic, they may check-in and receive forms to fill out. Some of the collected information may be biographical, medical information, and/or surveys to assess the patient. Examples of surveys for this condition are, but not limited to, Oswestry Disability Index (ODI), Short Form 36 Health Survey (SF-36), Neck Disability Index (NDI), Patient-Reported Outcomes Measurement Information System (PROMIS), and the like. These forms may be filled out digitally or on paper. The digital forms may be viewed through the system to a tablet or phone. The physical form may have markings on it to tie it back to the patient and identify the form type. After the patient has filled out the forms, they may go back to a clinic room for evaluation. The evaluator may take the forms and scan the forms into the system via file upload or linked external device to capture photos of the forms. The evaluator may log in to an online portal or app to access the system and enter in basic information such as provider and appointment details. The system then may provide a QR code that enables the entry of data via mobile phone camera to capture the associated record and automatically capture and assign the information to the patient record. The forms may be tied to the patient record and automatically processed to capture and digitize information. The forms may aid in providing basic patient information, drugs, background information, custom questionnaires, insurance information, history, and/or a medical survey like those mentioned above. The data may be processed via computer vision, optical character recognition, AI and/or machine learning for handwriting recognition. The forms may have predetermined fields of interest for capture to extract information. This information may be stored in the patient record and trended over time as well as provide context to data provided in the system. In addition, information, such as, but not limited to, pain reports, height, weight, sex, previous surgeries, and the link can be used to be used as an input in sensor calibration and processing. In addition, information over time, such as improvements or declines in results from patient surveys can be mapped to treatment selection and to static postures, dynamic movement patterns, muscle activity or any other signal captured by the system to create predictions for optimal patient care based on a weighted input of factors, feature identification, classification, regression, or any of the listed AI/ML techniques mentioned herein. After the forms are scanned, the examiner may ask the patient to perform a series of movements such as a timed up and go, short physical battery, balance, sit/stand and reach, walk, or any other movement desired. The examiner may have a pre-programmed set of movements or change the movements to be recorded via the GUI. The examiner may use a phone to scan a QR code that transfers a link that guides the desired studies and enables video tracking on a mobile device camera. If a webcam is present, a webcam can also be used attached to the main device. If the device is a mobile device that already contains a camera, the examiner can proceed through the capture process on the same device. The application or web app may take individual captures of the trials and tests of the patient movement. The app may identify different movement trials and classify them automatically without the need to switch tests and/or stop video (e.g., without user input). The patient may perform these movements with or without mobility aids. If mobility aids are used an analysis of effectiveness or proper use of the aid may be performed. A trial may be performed with and without the mobility aid for comparison. The data may be captured and processed. The results may be generated and provided in real-time or after a processing period. These results may be used in the further examination and treatment determination for the patient. The provider may decide more information is needed and the wearable system may be proscribed for long-term evaluation of the condition. The system may use results from the long-term tracking, the video collection, and the forms to improve tracking performance, make predictions, give feedback to the wearer or clinicians, or any of the other functions mentioned in the document.

According to some embodiments, by way of a non-limiting example, the system may be used for the treatment and assessment of geriatric or frail patients in or out of the clinical environment. The system may be used to capture metrics about the patient relevant to conditions or factors such as, but not limited to, fall risk, dementia, Alzheimer's, Parkinson's, frailty, independence capability, cognition, functional capabilities, medication management, release from hospital, general management of these patients, the like. The patient may come in for a check-up or evaluation. The patient may receive a tablet or computer linked to our system. The tablet may have questions for the patient to fill out to test memory or cognitive ability. The tablet may have the front camera actively capturing as the patient performs the designated task. The system may be tracking the facial expressions, eye movements and responses, hand movements of the patient using the front-facing camera. The system may also be tracking time related metrics for the questions as well as the user's interaction with the system, such as touching the screen or moving the mouse. The system may also be accessing the internal sensors of the device, such as accelerometers, gyroscopes, and magnetometers to assess the motion of the device itself. The system may be processing looking for motions such as tremors of the device while being held. The data collected may be processed and aggregated into a report along with normalized age comparative metrics. The video and other sensor readings may be processed locally or in the cloud. The patient may also be asked to write information. They may be asked to write with their finger, a stylus, physical paper, or any other means of writing. The system may receive input from this and record the data to be tied back to the patients record. Motions of handwriting can be tied back to cognition and to disease related progression metrics for disease such as Parkinson's. If the writing is done outside of the system, the data may be imported into the patient record, or the physical page may be scanned via a picture or video to capture the information. The handwriting may be segmented from the page and analyzed for features relevant to the patient including, but not limited to, micrographia, macrographia, ink utilization, bradykinesia, tremor, velocity, pressure, kinematic features, and the like. The data can be associated with the patient record, trended over time, compared, and screened for possible intervention or risk factors. After the patient completes these activities, they may be back to the clinical examination room and perform a recording of the gait and movements as they interact with the clinician. The clinician may view the data collected from the system and decide to issue a long-term monitoring device for the patient.

According to some embodiments, by way of a non-limiting example, the system may be used by a clinician treating a patient for idiopathic scoliosis or screening for scoliosis. The clinician may request an assessment to be performed at home that captures the progression of spinal curvature. This may be due to a desire to monitor the progression of the curve, select treatment, screen them for further evaluation, or to evaluate the effect of treatment such as bracing or physical therapy. A link or a notification may be sent to the patient of the request. The patient may have someone record a video using the link provided for them, or place the phone in such a way that the camera points in the direction of the patient, such as a tripod. The patient may be asked to stand in their normal standing posture. Images or videos may be captured of the patient from the front, back, and/or side to capture the spine, pelvis, and shoulders. The system may have the patient move around to capture dynamic motion of the patient. In addition, instruction may be given to have the camera move about in different positions in order to provide more context about the scene and/or enable algorithms to intuit depth, size, shape, scene information, or other data relevant to the collection. These algorithms for example could use simultaneous localization and mapping (SLAM) techniques and may even take in information from the camera system itself such as accelerations, angular accelerations, or magnetic field strengths in order to better produce depth or special context from the captured images. Analysis may happen in the cloud where the spine patient will be located in the image using AI semantic segmentation. A deep learning pipeline such as a trained visual transformer using an online method may track the spine and pose of the patient through a progression of frames. The spine will then be generated in space and may be registered to a previously taken CT, X-ray, or MRI image(s). The algorithm may trace the spine and capture features such as pelvic alignment and tilt, shoulder alignment, and tilt; coronal, sagittal, and transverse angles of the pelvis, lumbar, thoracic, and cervical sections; cone of economy, and/or any other anatomical measure relevant to the condition. In some embodiments, objects in the scene may be used to point out or trace anatomical features. This may be someone using a known stylus to trace the spine and point to regions of interest. It may also be someone's finger tracking the spine and shoulders. This may aid in the further identification, segmentation, and/or mapping of the patient. In some embodiments, a mesh may be created that captures the patient. This mesh may be combined with identified points of interest to be located and analyzed. These points of interest may be mapped and multiple points can be used to create lines or curves associated with the images. In some embodiments, marks known or unknown to the system may be placed or marked on the patient to aid in location and identification of key tracked points. This may be a sticker or a marker identifying the spine or individual points on the body. These marks may be known and used as guides for orientation, size, perspective, or other references in analysis. These markers may be a QR code or checkerboard of known size placed in the frame or on the patient. These markers may also reside not touching the patient, such as a ruler on the ground or a grid on the floor. This data may be used in the analysis of the images for detection or for frame context to increase accuracy of measure point locations in space. This data may be transmitted to the patient record and presented to clinicians. It may be trended over time and compared with other records in the system. It may be used to track treatment progress to improve or alter treatment course. It may also be used to plan proper surgical intervention or physical therapy interventions.

In some embodiments, the sensors can be placed on the subject and a tablet device (or other external UE, for example) can be used to capture video of the subject as they perform a series of range of motion exercises and/or a walking test. In some embodiments, the sensors on the subject can be connected wirelessly to the UE streaming IMUs and EMGs. In some embodiments, the streaming sensor's signal can be time synced with the video of the tablet as the video is recorded. In some embodiments, the recorded video can be analyzed either locally on the tablet or in the cloud. In some embodiments, such images can be segmented for the subject and the pose of the subject can be determined using an AI/ML (e.g., deep learning, for example) model.

In some embodiments, further analysis of the pose can be used to determine the joint angles of interest and the posture of the subject. In some embodiments, this can be performed over the course of an exercise and a walking test, for example. In some embodiments, a matched set of data from the time synced IMU data (e.g., acceleration, gyroscope, and/or magnetometer data, and fused measure of these inputs to produce the orientation of the device using Kalman filters, for example) and/or EMG signals, can be used to create a transfer function to the corresponding frame with a calculated pose estimation to enable accurate pose and joint estimation without need for cameras.

According to some embodiments, the UE (e.g., table) can provide an output (e.g., display, audible sound and/or haptic effect, or some combination thereof) that can indicate the success of the calibration. In some embodiments, the UE can output instructions to the user as they perform the different tasks, as well as provide feedback on position and camera position.

In some embodiments, instructions may be given to the user via an electronic display such as a tablet. These instructions may be in visual, haptic, or auditory feedback. In some embodiments, such instructions may be different tasks or movements. For example, instructions may be a video played on a tablet that shows the correct motion for the calibration or exercise. In some embodiments, the wearer of the device may be asked to follow along to these motions as they are seen on the screen. In some embodiments, an auditory signal from the system may play such as a tone to indicate when the patient should move. In another non-limiting example, instructions may be a mixed reality overlay (e.g., via a MR headset, for example) of the wearer of the device performing calibration movements and getting visual feedback as to the success of these steps for calibration.

In some embodiments, a volumetric model of the subject can be generated using the camera when they are calibrating. In some embodiments, such model can provide additional factor information, such as, for example, the size and mass distribution of the patient. In some embodiments, such model can be utilized for the creation of a digital representation of the patient (e.g., an avatar, for example), which can be utilized as part of the generated report (e.g., as in Step 418, discussed below—for example, an XR display that depicts movements collected and measured of the patient).

In some embodiments, one or multiple cameras may capture the wearer in order to create a model surface or volume of the individual. In some embodiments, such cameras may be standard optical cameras, infrared, lidar, depth sensing, and/or any other type of camera capable of capturing the subject. In some embodiments, the disclosed model may be a volumetric rendering or surface rendering of the wearer. In some embodiments, such model may contain key points along the surface of the individual in order to create a surface mesh. In some embodiments, the mesh may be generated using any of the disclosed AI/ML discussed herein—for example, segmentation techniques for computer vision, deep learning, machine learning, and/or other AI/ML techniques for the creation of the mesh.

In some embodiments, such cameras may also be used to measure aspects of the wearer in order to inform a virtual avatar or representation of the wearer. In some embodiments, the representations and models may be utilized in the data display or visualization. In some embodiments, engine 200's operation may be aided by the addition of an object with fixed or predetermined dimensions in order to determine the proper dimensions for the disclosed model. In some embodiments, they may also be used in the description and instruction of articular motions and movements for the user.

For example, a user may be in front of a video camera attached to a tablet moving about during calibration to capture the wearer's body while a ruler acts as a landmark of known size on the ground. In some embodiments, as the wearer moves around a deep learning model segments the wearer from the background and measures points in order to create a 3D mesh surface of the wearer's body. In some embodiments, after sufficient frames of the body have been captured and points have been collected, the model may be generated with the surface to represent the user as an avatar. In some embodiments, for example, an avatar may be manipulated by engine 200 to bend and move creating a digital twin of the wearer. In some embodiments, this may be used in the reports generated by the device to show clinicians the wearers motions throughout the day. In some embodiments, the model of the body may be used to capture and/or analyze changes in body composition, size, weight, height, and/or other metrics about the body. In some embodiments, these measures may be used to track changes in muscle and/or body fat. This may be a global measure and/or localized to specific regions of interest. In some embodiments, this may be used to track changes in size due to symptoms such as swelling, bloating, inflammation, the build-up of fluid (e.g., lymphedema or liver disease), wound healing, skin abscesses, skin abrasions, and/or other observable and/or measurable symptoms such but not limited to redness, skin marks, stretch marks, skin changes (e.g., striations or allergic reactions), changes in color, and/or changes in texture. In some embodiments, direct measures of size, shape, and/or volume may be calculated and correlated to changes in physical condition and/or disease state. In some embodiments, these measures may be taken during dynamic motion and/or analyzed over time.

In some embodiments, motions of the skin may be known, modeled, and/or measured to correct sensor readings such as motion, orientation, location, muscle signals, and/or pain measures. In some embodiments, this may be a model of skin movement with respect to bony structures based on X-rays, MRIs, CTs, computer vision, measurements using skin-based markers with respect to one another and/or to boney landmarks, measures of sensor motion with respect to calculated pose of the patient, and/or any other method of measuring skin motion. In some embodiments, patient information such as height, weight, age, or any other collected factors may aid in the modeling of skin motion. In some embodiments, these known and/or measured skin motions may be used to create transfer functions and or ML/AI models to account for skin motion with respect to anatomical positions.

According to some embodiments, by way of a non-limiting example, a model of skin movement taken from a data set using IR camera system and skin stickers with positional measures of anatomical marks may be taken for a set of patients in numerous postures and positions. The relative motion between markers and bony anatomy is calculated based on gradients of motion for different postures and dynamic situations with respect to the location of each position. The measures of patient BMI and age are used as inputs to the model of skin motion. The sensor locations are determined and transfer functions for the patients are created to correct orientations of the sensors to the underlying bony anatomy to achieve a better accuracy in body position and posture measure throughout the monitoring period.

In some embodiments, calibration can involve identifying a reference object with a known dimension so that capture imagery can have frames with known sizing measurements. For example, a ruler placed on the ground, a logo on a garment or sensor, and the like.

In some embodiments, an object of known dimension may be placed in the frame, near the where, near the sensor, on the sensor, on the garment, and/or on the wearer. In some embodiments, the frame may be captured by a camera or any other system capable of capturing the scene. In some embodiments, such object may serve as a reference in order to determine the true dimensions of an object captured in the frame.

In some embodiments, the garments and or the sensors have features, such as, for example, printed markings, reflectors, colors, and or geometric features that aid in the tracking during calibration. In some embodiments, such features may aid in the visual tracking of the sensors and/or the segmentation of the wearer. For example, such features may be black circles printed on a white garment that outline the wearer and trace the center of the back along the spine. In some embodiments, this may be utilized in the segmentation of the images captured by the camera in order to determine distinguished important landmarks. In some embodiments, such landmarks may aid in the calibration discussed herein by showing the spinal segments and shape of the wearer's body. In some embodiments, marks on the sensors may be QR codes or other unique markings such that the camera system is able to distinguish the sensors and determine their locations in the overall system or in reference to the wearer's body.

In some embodiments, parts of the calibration may run locally, and others run in the cloud. Data from the device may be uploaded to the cloud for calibration and pose estimation. In some embodiments, calibration processing may be after the offloading of the data from the system.

In some embodiments, calibration performed in Step 404 may be performed without the aid of a camera. In some embodiments, such calibration may be done through the wearer performing certain motions or movements upon instruction. In some embodiments, such motions and movements may be done directly on the sensors or done when the sensors are worn. By way of a non-limiting example, such calibration can involve a user with a sensor(s) on the legs receiving instructions to bend their legs at 90 degrees, full extension, and fully flat on the ground. In some embodiments, a reference guide may be used such as a plastic guide that has 90 degrees or other increments on it so that the user knows when they have achieved the desired positions.

In some embodiments, the sensors may be placed and another object may be used to determine the location and orientation of the system. In some embodiments, such objects may be detected using cameras or tracked using internal sensors. For example, a user can be using a stylus tracked using computer vision via a camera and object segmentation touching the sensor(s) in the system and other landmarks on the wearer.

In some embodiments, sensors and other parts of the system may need to be synchronized. In some embodiments, such synchronization may come in the form of a trigger or timing pulse(s), which can be predetermined and/or dynamically determined. In some embodiments, it may also be done digitally to set the time of each sensor to a reference.

In some embodiments, there may be an enclosure or tray to house the sensors. In some embodiments, the enclosure or tray may connect or couple to the sensor to provide charging to the sensors. In some embodiments, this may aid in the synchronization and/or calibration of the sensor system. In some embodiments, the housing may have access to the internet or wireless communications. In some embodiments, the system may connect to a computer or external system wirelessly or through a wired connection. By way of a non-limiting example, a user preparing to place the sensors may press a button on the housing to start the initialization. The housing may have access to the internet via a wireless communication module that provides 5G connection to internet. The system may fetch the current date and time. The housing through a wireless and/or wired connection to the sensors may communicate the date and time to the individual's sensors, and/or set a timing pulse so that the sensors have the synchronized timestamps. The devices may contain real-time clock components, crystals, and/or other mechanisms of maintaining time on the device. The housing through lights and audio may tell the user to set the housing on a flat surface motionless. The housing may send a pulse to the sensors wirelessly or through contacts in the housing communicate to calibrate the gyroscopes. The system may then flash a green light and indicate for the user to go on to the next step which requires the user to rotate the housing with the sensors attached in an instructed manner. The system may send another signal to the sensors inside to calibrate the accelerometers and magnetometers based on a dynamic motion calibration such as rotating the system in all axis of measure. The system may query the sensors to ensure all battery status, calibration status, and device function is positive. The housing may then indicate that the system is ready for use.

In some embodiments, the housing may contain tracking capability, such as GPS, Enhanced Inertial Navigation Systems, cell tower triangulation, and/or any method of location. In some embodiments, these systems are also embedded into the sensors of the device. In some embodiments, the housing may be used to track and manage the sensor systems, such as to keep inventory, find lost items, and the like. In some embodiments, these systems may be used in providing extra context to the system, such as location, altitude, activity level, location or distances traveled, and the like. In some embodiments, these systems may be utilized when tied in with other system context such as an appointment time and aid in the reminder or notification of the wearer. In some embodiments, these systems may be used to instruct the wearer of critical task, such as activities, appointments, and/or the return of the system. In some embodiments, the enclosure may connect to the worn system wirelessly to send or receive information. This information may be updated timestamps, instructions, exercises, or any other data that may aid in the collection. Information may also be received by the housing from the sensors, such as downloading information off of the worn devices and uploading this to the patient record. In some embodiments, the housing may be used to recharge the devices. In some embodiments, the sensors may be brought near or placed in the system and data transferred from the devices to the housing. The housing may store and may encrypt the data. The housing may also send this data to another system or upload it to the cloud. In some embodiments, the housing might connect to external devices. These external devices may be a phone, watch, tablet, UE, and/or fitness equipment in order to communicate information to these systems. In some embodiments, the housing might connect and communicate with external devices used to calibrate the system. This may be a phone recording a video as one non-limiting example. The data may comprise timestamps, device information, calibration information, or any other information used in the system. In some embodiments, the housing may program or transmit data about the setup or calibration of the system to the sensor devices. In some embodiments, the housing may contain instructions or information for use by humans or computers. The system may have written instructions, QR codes, NFC, RFID, Bluetooth, BLE, or any other communication mechanisms.

According to some embodiments, by way of a non-limiting example, a patient is sent a system for long term monitoring. The system may, upon startup, verify all of the sensors and calibrate the system. The patient may put on the worn devices and sensors. The patient may scan and/or tap their phone to the box and through NFC the system launches the app or URL with embedded system registration information. This ties the patient profile to the new system and may include the status of the system. The system may connect to the wearer's phone via BLE, and it may act as a UID or other communication device to the phone. It may transfer timestamp and sync information to the phone when capturing video data. This data may be stored in the housing memory, processed on the phone and/or transmitted to the cloud. In some embodiments, this data is then used to serve as the calibration for the worn sensor system. The housing periodically searches for the worn devices and receives data updates from the device, and such searches may take place nearly continuously in some embodiments. The housing may upload data to the cloud for processing and tracking.

In some embodiments, the user can also access the data collected via a mobile app. In some embodiments, the system may have a timer that indicates when the data collection process is completed, for example after two weeks and may send a notification to the user via email, text, push notification, or notification on the housing itself such as a light, sound, vibration or visual. In some embodiments, the housing may transmit data to the system to confirm the data collection and validate the data. The patient may put the sensors back in the housing and a notification may be sent to the system to alert that the sensors are ready to be shipped back. In some embodiments, the shipping information may be displayed on the housing. Or in the case where the wearer returns the device back to their doctor, the system may receive notification about the date and time of the appointment. In some embodiments, the housing may display that information and notify the patient with reminders of the appointment and reminders to bring the housing with the enclosed sensors to the appointment.

In some embodiments, one or more methods of synchronization between devices may be used. Synchronization may be in the form of trigger pulse, either physical or wireless, from the house, remote device, mobile device such as a phone or tablet. Additionally, the system may synchronize through light flashes on the devices captured by an external device or camera system. The system may be synchronized by cellular or wireless connection to a global time. The system may be synchronized by physical methods such as a tap, touch, or spike in movement measured by the sensors to create a known point in time used to create timestamps. In some embodiments, synchronization may happen through a mesh network of devices or multi-device connectivity that propagates through the system. Any known or to be known mesh networking functionality may be used, and each device can help extend range if needed by relaying information in some embodiments.

In some embodiments, the activity of the wearer can be analyzed and synchronized over time. In some embodiments, the system may calculate drift between sensors and correct timestamps to counteract individual sensor drift either live or in post processing. By way of a non-limiting example, the wireless sensor may all be placed in the housing. There may be a button on housing to start synchronization and calibration. The system may set the global time using internet connection and share that via a trigger pulse, wired, or wireless communication to the sensors. The sensors may be placed and begin recording. The sensors synchronization may drift over time locally on each device. The sensors may periodically come into signal reach of the housing and receive updated global time and save that to memory. After collection, in post processing, the data between devices may be analyzed looking for deviations between sensors and predicted versus true global time points. The system may use dynamic time warping, interpolation, time stretching or shrinking, or any other method to sync the data sets. Additionally, in some embodiments, analysis may be done to find features in the data to help further align the data sets such as steps or periods of activity and non-activity. Some embodiments may take the overall start time and known relative endpoints created by removal and attachment to the housing to adapt the data. A known start sensor removal timestamp and sensor reconnect timestamp to the housing may be collected and stored in the housing for correcting drift over time.

In some embodiments, the housing may contain, hold, or affix one or more cameras, IR cameras, ultrasound probes, optical cameras, infrared, lidar, depth sensing, and/or any other type of system capable of capturing the subject. In some embodiments, the camera system may just digitally connect to the housing such as through Wi-Fi, Bluetooth, or any other means of connection mentioned herein. In some embodiments, there may be one or more cameras, IR cameras, ultrasound probes, optical cameras, infrared, lidar, depth sensing, and/or any other type of system capable of capturing the subject part of the system without an attachment or connection the housing. In some embodiments, the housing may aid in the holding, steadying, positioning, or moving of the additional capture system.

According to some embodiments, by way of a non-limiting example, the housing may contain a multi-camera (depth sensing) array attached to an extendable pole or other suitable support device or surface that rises from the housing. The wearer may set this system up and point the camera in the direction of the calibration. Depending on the setting, the wearer may also extend legs from the housing to allow for proper collection angle without the need for a table or furniture to set the housing on. In some embodiments, the housing may record footage of the calibration with the corresponding timestamps. The housing may then process the footage and save the footage in memory. It may then upload the data to the cloud for further processing.

According to some embodiments, by way of a non-limiting example, the housing may hold a mount for a phone. The mount for the phone may have legs to stand the phone at a better viewing angle. The mount may have a clamp to attach to the phone. The mount may have a motor to enable tracking of the wearer. The wearer may connect their phone to the mount via Bluetooth. The phone, through an application, may analyze the video, locate the wearer, and send a signal to the motor through a control system to aim the camera at the wearer. In addition, the mount may also impart motion to the camera. This motion may be used in further video analysis, such as a SLAM analysis to determine 3D data from the scene and about the wearer. The movements of the system may be recorded and saved to memory and/or uploaded to the cloud to have motion from the mount in addition to the video data to aid in the video processing.

In some embodiments, one or more additional sensors may be added to the worn device, to the housing, or added as an addition to the system. These sensors may enable Wi-Fi Positioning, Bluetooth Low Energy (BLE) Beacons, Ultra-Wideband (UWB) locating, Ultra-Wideband (UWB) direction finding, Bluetooth Beacons, Bluetooth Direction Finding, Radio-Frequency Identification (RFID), Radio Frequency pose, Radio frequency directional finding, Acoustic Positioning Systems, Magnetic Positioning Systems, or any other radio frequency locating, position finding, pose, or tracking method. In some embodiments, the UE may contain these technologies listed above. In some embodiments, these technologies may be used to capture signals and perform analysis such as Time of Flight (ToF) Measurement, Pulse Repetition Frequency (PRF), Channel Impulse Response (CIR), Angle of Arrival (AoA), Angle of Departure (AoD), and Time Difference of Arrival (TDoA) to measure the signals from the system. The raw signals or the calculated measures may also be further analysis by ML/AI systems to aid in analysis. The data may also be combined with any of the data collected by the system.

According to some embodiments, by way of a non-limiting example, the worn sensors, housing, and mobile smart phone may contain UWB. The worn sensors and housing may transmit and receive the UWB signals, they may use Time of Flight (ToF) Measurement, Pulse Repetition Frequency (PRF), Channel Impulse Response (CIR), Angle of Arrival (AoA), Angle of Departure (AoD) and Time Difference of Arrival (TDoA) to measure and calculate distances and locations with respect to one another. These distances may be used to calibrate the system or in tracking. These measures and calculations may also be aided by data from other sensors in the system, such as the accelerometers, gyroscopes, and magnetometers of each device. These signals may be interpreted and synchronized in such a way that the position and orientation of each sensor with respect to one another and the housing is known. In some embodiments, the housing may act as a global reference frame for the system in the analysis.

According to some embodiments, by way of a non-limiting example, a WIFI or other RF system or device may be embedded in the housing. In some embodiments, this system may transmit RF signals and these signals may be reflected by the wearer and other objects in space. In some embodiments, these signals can be received and interpreted through any number of ML/AI systems in order to track the pose and/or position of the wearer with or without the sensors. The sensors may be worn at the same time and tracked with timestamps that can be related to the RF reflections in order to capture the pose and posture of the wearer. Additionally, these RF reflections can be used to track other physiological measures such as respiration or heartrate and this data can be utilized by the system.

In Step 406, engine 200 can perform sensing operations as discussed herein. According to some embodiments, sensing of Step 406 can correspond to the time in which data is collected. Data may be collected over a matter of minutes and/or days. In some embodiments, sensing time may be included as the time during the calibration phase. In some embodiments, during the sensing time, certain sensors can be active in the data collection and others may not be active. In some embodiments, the sensing array may be worn with all sensors attached to the wearer, or in certain cases, sensors may be removed from the array.

In some embodiments, the sensing array may be worn by a patient who is being evaluated for medical treatment. For example, a patient may be experiencing back or leg pain. In some embodiments, the array may be worn for a predetermined period of time (e.g., 48 hours, for example), which can enable the recording of the patient's daily motion and activity. In some embodiments, some of the sensors may be taken off for sleeping.

In some embodiments, the sensing array may adjust sampling rates depending on the motions sensed or the optimal values for the condition and patient. The system may even sleep, turn off, or stop recording data from sensors depending on the state of the device. In some embodiments, the activity of the patient may be determined, and this may be used to drive changes in the sensors collected and the rates of these sensors. In some embodiments, the sensing array may communicate with one another to trigger the changes in sensing. In some embodiments, the system may dynamically update the sampling rate due to sensed noise or a sensed trigger. These triggers may be a sudden change in movement, activity status, muscle firing, location, time of day, or any signals from other sensors in the system such as heart rate, respiration, and/or any other signals mentioned in this disclosure. In some embodiments, the signals may be captured at high frequency and recorded to memory at a different frequency depending on events and/or data features of interest.

According to some embodiments, by way of a non-limiting example, a sensor system may be worn for a two-week monitoring period. The system may aim to save battery and storage space by adjusting sample rates and sleeping sensors. The system may have the ability to analyze gyroscope or accelerometer data live on each device in order to determine when the wearer is in motion or static. It may also be able to classify the movements into categories such as walking, sitting, lying down, running, biking, exercising, or any number of activities. When the wearer is sitting down the system may choose to only have only one of the sensors in the array active and the rest of the sensors sleeping. When the accelerometer of the monitoring device senses a change in motion, it, through BLE mesh, indicates to the other sensors to wake up and start sampling at a low rate. The sensors may monitor the movement and determine that it is likely the wearer had transitioned into a standing position and started to walk. The sensors in the mesh may decide to increase the sampling rate on all sensors and to turn on the EMG sensors and record data. The system may sample until there is another change in activity, such as standing still, where it will reduce the sampling rate and saving rate of the sensors.

In some embodiments, one or more networks between devices or to other devices may be established. These networks may be Bluetooth, Bluetooth Low Energy, Wi-Fi, NFC, RF, or any other wireless or wired communication method. In some embodiments, the networks may be Point-to-Point (P2P), Star Topology, Mesh Networking, Message Hopping, Hybrid Networks, Body Area Network, Peer-to-Peer (P2P) Network, Gateway-Connected Network, Broadcast in Mesh Network, Group Addressing in Mesh Network, Publish-Subscribe Network, Data Concentrator in Mesh, or any other network created to communicate between devices. In some embodiments, these communications may be used to offload data processing, share processing load, reduce battery consumption, reduce memory or consolidate memory, detect device function and alert if there are issues, monitor the health or battery status of the sensors, connect external sensors to the system, communicate data for analysis purposes, extend the range of the signal, aid in time synchronization, and/or other functions useful in the system. In some embodiments, multiple sensing arrays and systems for multiple wearers may be connected to provide context and further analysis. In some embodiments, by way of a non-limiting example, a military squad or soccer team may have multiple systems in use. The systems may connect to one another in order to provide coordinated feedback for the group such as optimal locations or body positions. These systems could provide classifications of motions such as defensive or offensive posture and provide feedback. The system may use the mesh network to strengthen the signal or provide network stability.

According to some embodiments, by way of a non-limiting example, the sensor arrays establish a BLE mesh. They may periodically send status updates. In some embodiments, the system may determine that one sensor has become loose and could potentially fall off. The system may notify the user through an audio recording on the device nearest to the ear. In some embodiments, if the device is not fixed, the system may through connection to the wearer's phone send a notification to the wearer with instructions on how to correct the error.

According to some embodiments, by way of a non-limiting example, the sensor arrays establish a BLE connection between sensors using a many to one network. The wearer may be running a marathon and is interested in detailed running information such as posture and running form. The system may additionally connect to wearer's headphones and watch. The sensors may send data to one of the devices to analyze and interpret data readings from the sensor and run analysis. The data may be transmitted to the watch, where more processing occurs, and the data is relayed to the runner. The system may also connect to another external device, such as a smart knee implant. This implant may be providing force data on the knee replacement which is analyzed by one or more sensors in the array. The system may also be connected to a heart rate monitor and this data may be recorded and analyzed for the system in a time synchronized manner to give feedback to the runner. This feedback may be about ways to optimize strike length and posture in order to reduce knee forces and optimize exertion.

In some embodiments, the system may connect to other devices such as smart implants, skin-based sensors, medical equipment, workout equipment, or other sensors or systems in the environment or on the wearer. In some embodiments, these sensors may be connected to the system using any number of communication methods such as BLE, NFC, RFID, Medical Implant Communication Service (MICS), UWB, Inductive Coupling, Zigbee, or any other communication systems known to communicate to the system. In some embodiments, the wearable device may act as an interrogator to measure signals from the device, such as sensing magnetic field, voltage, capacitance, optical methods, ultrasounds, and/or acoustic sensing. In some embodiments, the system may act as a recording device to capture data from these systems. In some embodiments, the system may take in data and add to the processing or analysis of the data. In some embodiments, context from these systems might trigger or alter function of the sensing array or the system. In some embodiments, the wearable array or the system may send signals to influence or change the other system.

According to some embodiments, by way of a non-limiting example, the sensing array may connect to an implanted neurostimulator. The sensing array may be streaming data from the simulator and processing it locally. The sensing array may detect a motion that has previously induced pain in the wearer and instruct the neurostimulator to modulate the signal to inhibit the pain pathway. After time of wearing and tracking of neurostimulation the system may upload data to the cloud where analysis may be done to optimize the neurostimulator frequencies, locations, and amplitudes based on the data collected from the system.

In some embodiments, during the sensing period, a microcontroller can monitor the patient's movement to determine the activity status of the patient. In some embodiments, accelerometer, gyroscope, magnetometer, EMG (and/or any other type of muscle sensor device, technique or technology, whether known or to be known), and/or barometer data from the sensors may be used to classify the activity of the patient, such as exercising, walking, standing, sitting, lying down, or sleeping. In some embodiments, such classifications may change attributes about the data collection, such as, for example, collection frequency or which sensors are being collected from. For example, when the wearer is lying down, the wrist and lower back sensors are sampled at a low frequency to conserve storage and battery.

According to some embodiments, Step 406's sensing can involve pain collection. In some embodiments, such pain collection can be integrated into UE 102 and/or the applied sensors (from Step 402), and in some embodiments, can be utilized by an additional device (or some combination thereof).

According to some embodiments, a component of sensing may be the data collection of pain signals. Pain tracking refers to the collection of information about pain through a reported measurement, automated interpretation, or hybrid form of collection. An input device can be a UE—for example, a watch, tablet, pager, device, or built into the garment. In some embodiments, the location of pain may be reported to any designated region of the body including the head, torso, limbs, joints, muscles, and the like. In some embodiments, inputs from the user may be provided through touch (button, screen, surface), voice, or motion.

According to some embodiments, by way of a non-limiting example, a pain monitoring device may come in the form of a device that sits behind the wearer's ear. For example, in some embodiments, the device affixes to the skin through adhesives. In some embodiments, the device may contain a button, contact, a pressure sensor or another conventional sensor or actuation device for the wearer to interact with the device. Upon interaction, in some embodiments, the device's speaker can transmit a noise via bone conduction to the wearer to indicate the start of the interaction. In some embodiments, the device can contain a microphone to record the voice of the wearer for an interaction. In some embodiments, the wearer may tell the device to record a pain score along with a location and activity that caused the pain. In some embodiments, the device may record the signal to store the data or process it locally on the device. In some embodiments, the device may store the information in memory or transmit it to a nearby receiver. In some embodiments, the device may be placed on the posterior auricular vein and use PPG to detect heart rate. In some embodiments, the device may also contain IMUs to record orientation and acceleration for use in determining patient activity or position. In some embodiments, device may also prompt the user to record an input on status. In some embodiments, the recording may be a recording of a physician they know to increase compliance. In some embodiments, the voice recording may be generated through voice recording or synthetic voice generation. As just one example, AI/ML models can synthetically create voices that match a recorded example. I.e., if a physician records a couple of phrases, the recording can be run through the system in order to make all the desired synthetic voices match the physician's voice.

In some embodiments, a pain recording device may be placed on the trunk, arm, in proximity to the ear, or held in the hand of the user, and/or other portions of the anatomy of the patient.

In some embodiments, with regard to pain metrics, pain may also be designated to a subclassification of the body part, such as, for example, the curvature of the spine or joint of a finger and may be specified with data referring to the location of pain, depth of pain, side of the pain, the severity of pain, the start point of pain, the endpoint of pain, perception of pain, path of pain, description of pain, duration of pain, and breadth of the pain, and the like. In some embodiments, the reported information may also include the level and severity of the pain. In some embodiments, the information derived from pain tracking may be utilized in combination with any additional metric including, but not limited to, postural analysis, muscular activation, gait analysis, activity monitoring, and physiological measurements, and the like. In some embodiments, the combination of the inputs may be used in the categorization or analysis of pain. In some embodiments, it may also be used in the combination and creation of the outputs.

In some embodiments, pain tracking may require input from the user. In some embodiments, the user may enter the data manually into the array, a device associated with the array, or directly onto the array itself. Inputs from the user and the system may be stored in memory and/or process on the array. In some embodiments, such information may be used to enable logical driven assessments of pain. In some embodiments, this may prompt the user with a survey of their pain.

According to some embodiments, using programed logic, pain assessment may prompt the user during a time of day, a specific activity, a muscular activation, or a change in sensor inputs. In some embodiments, as pain tracking is collected, engine 200 may assess the inputs to determine more timely prompting of questions for the user. In some embodiments, the inquiries/questions of the user may also change in response to the inputs from the system. In some embodiments, a logical assessment may be performed to validate a specific observation or report of pain. In some embodiments, a logical assessment may also be used to reduce survey fatigue by altering the questions, cadence of questions, or trigger of questions. In some embodiments, pain tracking may be automatically collected at random intervals, certain times of day, or based on logical assessments and changes in sensor inputs.

In some embodiments, logical assessments may also be used to help further assess the condition in greater detail, such as asking questions in situations that may be a contraindication of pain or in other situations when a similar pain may be likely. In some embodiments, engine 200 may determine if/when a certain posture or pose increases or decreases the pain level through the analysis of other metrics. In some embodiments, the input of activity and pose may be used in conjunction to determine the causes of pain. In some embodiments, EMG sensors (or other muscle sensors herein) may be used to detect changes in muscle patterns or usages such as over-activation, fatigue, twitching, spasms, non-symmetric usage, or other measures of the muscle in combination with pain-sensing or to trigger a response from the user about their pain. In some embodiments, pain tracking may be prompted or recorded utilizing user inputs including touch or voice activation.

In some embodiments, pain may be detected using inputs from the sensors of the array. In some embodiments, this may substitute or supplement the information manually input by the wearer. In some embodiments, it also may serve as a form of validation of pain or correction for wearer pain levels. In some embodiments, engine 200 may utilize physiological measures, such as heart rate, breathing, perspiration, skin temperature, electrodermal, pupil response, or other physiological metrics in order to sense pain or the likelihood of pain. In some embodiments, additionally or alternatively, abnormalities in posture, activity, gait, and muscle activity may be used to determine possible pain events. In some embodiments, additional sensors on or connected to the array may also aid in the detection and discernment of pain, such as a microphone capturing pain events and quantity through audio activation. Additionally, in some embodiments, other contexts may be captured to better discern the information collected, such as the location of the event captured through GPS or other means of location information. Additionally, in some embodiments, the time of day, temperature, and other factors describing the state of the wearer may be used in the analysis process. In some embodiments, this information may also be utilized in locating the source of pain and determining the level of pain disruption. In some embodiments, the combination of the systems may also be used in creating a score or weighting system for the analysis of captured pain metrics.

In some embodiments, audio recordings may be captured from the user. In some embodiments, such recordings may be the voice of the wearer and/or external noises. The voice may be analyzed for its content and/or its audio features. In some embodiments, sentiment analysis may be used for the recordings. Other forms of vocal analysis or speech analysis may also be used. In some embodiments, recording may be utilized to analyze emotional factors of the wearer such as depression, anxiety, and/or cognitive ability. In some embodiments, these recordings may be used to monitor the state of disease, such as Parkinson's, stroke, Alzheimer's, amyotrophic lateral sclerosis (ALS), MSA, or other neurological conditions. In some embodiments, these recordings may be used to provide a score for different factors of the tracked condition. These scores and recordings may be tracked over time to provide information about the wearer and aid in the analysis. In some embodiments, recordings may be used for classification of activity. Recordings from the environment may be used to contextualize and classify activity. In some embodiments, recordings may be used to determine exertion, effort, respiration, coughing, wheezing, heart rate, or other physical factors capable of being tracked with audio recordings.

According to some embodiments, the disclosed framework (e.g., and/or UE 102/sensor 112, for example) may be utilized longitudinally over an extended period of time. In some embodiments, this may allow for relative tracking over the course of a patient's treatment, diagnosis, or chronic care. In some embodiments, this may allow for finite testing to measure compliance, proper positioning, and posture while performing activities. In some embodiments, data from the longitudinal usage may be incorporated into the analysis of the device. In some embodiments, this may be interpreted to make decisions of a course of treatment, medication, sports rehabilitation, approved activities, progression or regression of a condition, or any other perceived metrics by the user and the object. In certain embodiments, the object profile may be saved onto the device for updating and revisiting.

In some embodiments, the disclosed framework can use the sensor inputs in order to classify the wearer activities and motions. In some embodiments, as discussed herein, activity monitoring refers to the classification of a performed activity by the subject at different instances. In some embodiments, the mentioned activity may refer to, but is not limited to, standing, walking, running, lying down, sitting up, sleeping, swimming, or any other activity a subject may perform throughout the day. In some embodiments, the activity may be qualified with metrics pertaining to duration, frequency, quantity, and quality.

In some embodiments, the disclosed framework may contain features in order to determine proper sensor placement and attachment. In some embodiments, engine 200 can utilize such measures in order to determine the proper placement of the sensor when the device during placement or during the course of the sensing process. In some embodiments, there may be other means of determining sensor detachment such as an anomaly detection algorithm to aid in the determination of data reliability. In some cases, the sensors 112 may move during patient usage (after initial sensor 112 placement). This movement can be noted on the report if not corrected throughout the usage. In some embodiments, engine 200 may notify or alert the wearer via sound or vibration of the detection in order to correct the issue.

In some embodiments, the worn sensors may contain several capacitive sensor electrodes to monitor conductivity and determine skin contact. In some embodiments, such sensors may be placed on the upper edge of the sensor or distributed across the skin side of the sensor to determine if the device starts to fall off the wearer.

In some embodiments, the worn sensors may have temperature sensors that measure the temperature of the bottom of the sensor. In some embodiments, such temperature can be normalized using a second temperature reading on the upper surface of the sensor. In some embodiments, the temperature differential may be used to determine if the sensor has become detached from the skin.

In some embodiments, engine 200 can assess the movement of one sensor in regard to the motion of other sensor(s) in the system to determine if the sensor has become detached from the patient. In some embodiments, the motion of one sensor regarding the system can be used in shape comparison or statistical determination of the measured device variance. In some embodiments, individual sensor motion may be characterized and measured with regard to frequency analysis, magnitude, noise, vibration, or other motion characteristics to identify malfunctions in the sensor, adhesion, or placement. These signals may be analyzed for changes over time at aid in the identification of these conditions.

In some embodiments, the worn sensor may contain an EMG which may continually monitor the signal quality to determine sensor attachment or placement.

In some embodiments, according to some non-limiting example embodiments, a pain sensor may be located on the back of an ear (or other body part) via the addition of PPG and/or ECG for heart rate tracking of the pulse on the posterior auricular vein or retromandibular vein. In some embodiments, certain sensors may require a timing and/or synchronization pulse(s) to ensure syncing between sensors of the array.

In Step 408, engine 200 can effectuate the performance of user instruction. According to some embodiments, the wearer can be instructed to perform a number of tasks when wearing the system. In some embodiments, such tasks can include certain stretches to test range of motion, certain compound movements to test patient balance and mobility, and/or a walking test to track gait parameters and posture. In some embodiments, the tests can be used to create a score for the use in clinical settings or as an informative metric for the wearer. In some embodiments, when performing these tasks, a video may be simultaneously taken to record these events. In some embodiments, there may be instructions given through a tablet or other digital devices to display instructions for the tasks. In some embodiments, the user may also see a live representation of themselves during these tasks to help in the instruction process.

In some embodiments, a walking test is performed by the wearer. In some embodiments these may occur multiple times throughout the course of wearing the sensors and comparisons of the data may be used in the analysis. In some embodiments, during the course of the walking test, the posture of the spine is monitored. In some embodiments, segments of the spine may be monitored in order to determine changes in angle or motion. In some embodiments, gait metrics may also be collected to provide insights into the health and mobility of the patient. Additionally, in some embodiments, muscular activity may be collected in order to determine muscle patterns and fatigue of the wearer. In some embodiments, other physiological parameters, such as heart rate, respiration, blood oxygen, and other parameters may be taken into account during the testing to provide insight into the health and fitness of the patient.

In some embodiments, the sensor array can be composed of one or more components that provide input via the sensors. In some embodiments, the sensors can provide both static and/or dynamic data to be analyzed by the system. In some embodiments, engine 200 may take components of the input to provide interaction with the users and the object. In some embodiments, such interactions may be prompts, notifications, or instructions to solicit an action, input, posture, or feedback. In some embodiments, such notification can come in the form of vibration, sound, light, or display notification.

In Step 410, engine 200 can perform instructions related to live monitoring. According to some embodiments, before, during, or after the sensing period, the wearer or another party may wish to view the outputs of the system. In some embodiments, this may come in the form of an application. In some embodiments, such application may provide visuals or graphics in order to inform the wearer or other party. In some embodiments, data may be transmitted to an external device via a wireless communication method or cable in order to provide this display. In some embodiments, data from the device may be transmitted electronically and uploaded to a server enabling remote access to the sensor data.

In some embodiments, data from the device may be utilized by medical professionals in order to assess for dangerous or emergent conditions. Examples of this can come from surgical instrumentation failure, impingement of the spinal cord, nerve(s), or severe instability in joints, and the like.

According to some embodiments, by way of a non-limiting example, a football player recovering from a knee injury may use the system as method to analyze motions to determine if the player is clear to play by looking at muscle signals and motion signals in relation to particular movements or exercise. The same player may also wear the sensor array during practice in order to monitor motions to prevent the player from overloading the injured body part, such as the knee. The player may be alerted when they are overbending or out of alignment. Additionally motions and muscle signals may be used to determine muscle fatigue to pull the player from practice before risking another injury.

In some embodiments, the device may be used to determine the mobility of a patient. In some embodiments, certain events, such as, for example, falls, instabilities or imbalances, may be recorded. In some embodiments, such events may be transmitted to caretakers in order to inform treatment.

In Step 412, engine 200 can effectuate removal of the placed UE/sensor (from Step 402). According to some embodiments, after the completion of the sensing period (e.g., Steps 406 and 410), the applied sensor array may be removed from the wearer. In some embodiments, after the removal of the sensors, the data from the system may be offloaded from the devices (and, in some embodiments, for example, shipped back to the provider of these sensors).

In some embodiments, the sensors may be reusable—therefore, in some embodiments, there may be disposable components for the attachment or other coupling of the device. In some embodiments, casings and/or housings of the sensors may need to be replaced, as is typical with other types of medical devices.

In some embodiments, the sensor casings may be separate components. This may be for temperature, sizing, spacing, or usability. In some embodiments, the battery may be located separate from the hardware. This may be useful to help with the size and shape of the sensors. This may also be done to allow for easy access to the battery or to replace the battery for the system. This may allow for the sensors to remain watertight while allowing for the battery to be changed. In some embodiments, sensors may be separated from the rest of the hardware components, such aa microphone, IMU, temperature sensor, PPG, Pulse Ox, or any other sensor listed in this disclosure.

Continued with Process 400, Step 414 can be performed by engine 200, which can involve the upload (or storing, sharing, communication, for example) of the data collected during the sensing period and live-monitoring. In some embodiments, data may serve as inputs for the disclosed analysis of the collected metrics/data. In some embodiments, information that describes the wearer can be identified, which can include, for example, age, sex, height, weight, medications being taken, diagnosis or potential diagnoses, region or location of the array, biological and physiological measures, date and time information, and treatments already performed. In some embodiments, other information may also be provided in the form of, but not limited to, medical records, X-rays, MRIs, and CTs.

In some embodiments, additionally or in the alternative, data may be collected utilizing motion capture or image analysis in order to provide information to the array and the analysis for further calibration, measurement, and assessment. In some embodiments, such data may be manually input or imported from other sources. In some embodiments, data collected by engine 200 may be stored locally on the device for the duration of data capture and/or transmitted periodically to external storage. Transfer of the data can be done wirelessly or through cable connection. In some embodiments, data from the device may be uploaded to the cloud for processing and storage.

In Step 416, engine 200 can effectuate and/or perform the computational analysis of the collected and/or uploaded data. In some embodiments, such computational analysis can involve parsing, extracting, analyzing and/or determining metrics and/or information via any of the disclosed AI/ML techniques discussed herein.

According to some embodiments, engine 200 and the related components may be utilized longitudinally over an extended period of time. In some embodiments, this may allow for relative tracking over the course of a patient's treatment, diagnosis, or chronic care. In some embodiments, this may allow for finite testing to measure compliance, proper positioning, and posture while performing activities. In some embodiments, data from the longitudinal usage may be incorporated into the analysis of the device. In some embodiments, this may be interpreted to make decisions of a course of treatment, medication, sports rehabilitation, approved activities, progression or regression of a condition, or any other perceived metrics by the user and the wearer. In some embodiments, the object profile may be saved onto the device for updating and revisiting.

In some embodiments, these pain metrics may be utilized in determining the probable pain generators and used to determine the course of treatment. In some embodiments, metrics may also serve as a tool in order to communicate in some settings including but not limited to medical settings, rehabilitation settings, or use therapy settings.

In some embodiments, data may be inputted into the system to provide optimization for the context of the underlying system, the status of the wearer, the placement of the sensor array, prediction of metrics, or other supplemental device functions. In some embodiments, a medical diagnosis, initial symptoms, or initial observations may be provided. These may be analyzed through natural language processing (NLP) and/or AI/ML techniques including, for example, large language models (LLM) to provide the system data for treatment planning or assessment of outcomes. In some embodiments, data to provide information about the underlying system, such as X-rays or medical images, may be provided to extract measurements, relations, and possible motion paths for the system. In some embodiments, engine 200 may use one or more medical images to determine likely motion paths or be used in order to relate the measures of the system to the underlying anatomy.

In some embodiments, the computational analysis may factor in, but is not limited to, motion, posture, activity, compensation, and pain prediction and processing through AI/ML (e.g., deep learning) techniques. In some embodiments, analyzed data may include summarized positioning and activity data displayed in table, numeric, or figure format. In some embodiments, the disclosed analysis may create a score for usage by the user to interpret the overall status of the wearer. In some embodiments, this score may be compared against previous readings, a generalized standard, or may be interpreted for other uses. In some embodiments, pain tracking may result in a score of pain pertaining to a region of the body.

In some embodiments, such score(s) may be computationally compared to an individual, a standard generalization, or utilizing other features to contribute to an overall health score. In some embodiments, the metrics collected from the system may be used to compare the wearer before and after a period of time. In some embodiments, this period of time may be before medical treatment and after medical treatment. In other cases, it may be before physical therapy or after physical therapy. In some embodiments, collected metrics may be utilized to create an individualized baseline of patient mobility, activity, posture, pain, and muscle activity. In some embodiments, the data may be analyzed and comparative study to add relevance to the recorded metrics and determine trends of the wearer's metrics toward a goal.

In some embodiments, posture may be measured in conjunction and/or analyzed with gait. In some embodiments, the posture of the object may be taken in relation to phases or changes in gait. In some embodiments, this may be used to determine anomalies in motion and movement of the user. In some embodiments, this may also be used to determine the state of the users. In some embodiments, this may be used to track progress of a treatment path. In some embodiments, this may also be used to find changes in the patient's pathology from the previous measures. In some embodiments, in addition to and/or in the alternative, it may be used to track nerve function or changes in motion due to the underlying condition or physiology of the user. In some embodiments, this may be used to indicate deterioration of patient condition. In some embodiments, deterioration of the condition may include failure of surgery, failure of hardware, changes to pathology, and/or nerve damage.

In some embodiments, the measures of posture or posture in conjunction with other measures may also be used to determine or predict the motion of underlying structural, motion, or forces of the object. In some embodiments, engine 200 may be implemented to determine the motion or locations of bone or muscles of the user. In some embodiments, this may be achieved through the use of transfer functions, and/or AI/ML techniques, simulations, physics principles, kinematics, and inverse kinematic techniques, or some combination thereof.

In some embodiments, posture measures may be used in conjunction with measures and analysis of pain. In some embodiments, this may be used to improve the reporting or detecting of pain. In some embodiments, movements or postures may be associated with pain in triggering or relieving pain. In some embodiments, such measures can be utilized to investigate treatments or causes of pain, both in the context of the activities and postures that generate pain signals and in what pathology or diagnostic testing causes the underlying pain.

In some embodiments, motion analysis techniques, kinematics, simulations, and/or AI/ML analysis techniques may be used to detect and determine features of posture in relation to pain. In some embodiments, relations of posture to pain may be analyzed to create a score to be utilized in the assessment and analysis of posture features or impact on pain. In some embodiments, some scores may relate to the likelihood of different pathologies, locations, or disorders that may generate motion patterns or pain. In some embodiments, applications, such as determining disability or validating the pain, motion may be used to validate or predict likelihood of pain being from a particular injury or pathology.

In some embodiments, posture may be measured and/or analyzed in conjunction with muscle activation. In some embodiments, this may be used to determine anomalies in motion and movement of the user including compensatory mechanisms. In some embodiments, this may be used to track progress of a treatment path, recovery after an operation, or strength and conditioning of the object. In some embodiments, this may also be used to find changes in the patient's pathology from the previous measures. In some embodiments, in addition to and/or alternatively, it may be used to track nerve function or changes in motion due to the underlying condition or physiology of the user. In some embodiments, this may be used to indicate deterioration of patient condition. In some embodiments, deterioration of the condition may include failure of surgery, failure of hardware, changes to pathology, and/or nerve damage.

In some embodiments, the change in posture over time may be analyzed with activity, muscular fatigue, compensation, or other metrics. In some embodiments, such analysis may be interpreted to make judgments of recovery, training, and capability of performing certain tasks. In some embodiments, this analysis may be compiled with location, time of day, and duration metrics to provide context for the measurement and to better inform the analysis.

Accordingly, in some embodiments, the analysis discussed herein can involve and/or be based on, but is not limited to, muscle symmetry, muscle spasms and/or pelvic parameters, as well as any other type of known or to be known anatomical markers.

And, in Step 418, engine 200 can perform the data review and execute steps to create the report, as discussed herein. In some embodiments, the data collected and analysis created from this device may be utilized in, but not limited to, the following settings: inpatient clinics, out-patient clinics, rehabilitation settings, physical training or therapy, and personal usage. In some embodiments, the sensor system may be utilized for short-term or long-term measurement.

According to some embodiments, the functionality of the reported analysis and generated reports may be uploaded to a cloud and utilized through an electronic medical record, personal app, or distributed through email, text, or paper report. In some embodiments, the sensor system may be utilized in combination with clinical imaging platforms to optimize placement, track changes, or to generate conclusions and interpretations of the data.

In some embodiments, the generated and created data may be used in the following use cases: pre-operative patient analysis or tracking; post-operative patient tracking and analysis; compliance of patients under clinical settings for measurements of activity levels, performed tasks, and physical therapy; surgical determination; surgical planning; generation of patient scores for indexes or surveys such as, for example, a disability Index; user physical rehabilitation; sports training; tracking of progress of physical training; analysis of workout progress; safety measures while performing physical tasks; risk score creation of tasks; and measurements of impairment or inability to perform certain tasks. In some embodiments, such data may be utilized to create risk scores for users which can be applied to clinical, employer, legal, or trainer decision-making, for example.

In some embodiments, such score(s) may be computationally compared to an individual, a standard generalization, or utilizing other features to contribute to an overall health score. In some embodiments, the user may utilize the system to establish thresholds of healthy ranges for collected and calculated scores and metrics. In some embodiments, such thresholds may be used as goals for treatment, rehabilitation, or training. In some embodiments, the ability to match scores or thresholds over a time frame may be used for eligibility of treatment, fitness for a procedure or surgery, prescription of medications, return to work determination, insurance payments, or other general health and wellbeing parameters.

In some embodiments, the output may be depicted in a digital form or physical form and may not be directly associated with the array. In some embodiments, the digital depictions may include, for example, a website, electronic medical record, audio or sounds, application, virtual reality interface, augmented reality interface, or any other digital interface. In some embodiments, the physical form may be any type of printed media, 3D model, or any feasible physical representation of the output. In some embodiments, the output may vary depending on the user and may be customizable to the user's preference. In some embodiments, the user may interact with the digital interface to display information desired based on learning, communicating, monitoring, diagnosis, planning, teaching, or training objectives. In some embodiments, the interface may also augment elements in the physical or virtual environment to aid in the objective of the system. In some embodiments, the digital interface may allow the user to define characteristics of the object to aid the analysis. In some embodiments, input in the digital interface may change or update the analysis to create the output.

In some embodiments, elements of the digital interface may be customizable by the user. In some embodiments, a website may be used to display feedback to the user. In some embodiments, the website may allow the user to select which plots to display as they manipulate the data. In some embodiments, the website may contain input fields for a user to select a value for force applied, position, posture, pain or other characteristics.

In some embodiments, the digital interface may be any number of different means of providing feedback such as applications, virtual reality devices, augmented reality devices, tablets, laptops, phones, or any electronic device capable of providing feedback to the user. In some embodiments, the display of the information to the user could be any form relevant to the subject or objective of the intended for the objective. In some embodiments, the displays may be interactable to allow for analysis and viewing of different times, ranges, positions, and sub-characteristics (muscle groups activation, and the like) of the object. In some embodiments, data summarization may be made to allow for printing and physical analysis. For example, this may be any form of physical data representation including paper or 3D-prints.

In some embodiments, the digital interface may be used to categorize and add tags to the data. The tags man be manually created or automatically created. They may be automatically created based on age, condition, x-ray, surgery type, gender, or any number of data points captured by the system. Data points may be imported or taken from other software to create categories or tags. In some embodiments, data may be run through natural language processing and/or large language models in order to generate tags. In some embodiments, based on the data collected, tags, categories, or any other metric there may be analysis done to find similar patients or wearers based on the collected data. This may be done to aid in the diagnosis and treatment of the wearer. It may also be done to create predictive models for outcomes and treatment paths.

According to some embodiments, by way of a non-limiting example, a physician may be interested in creating a research study of spinal deformity patients. They may create a category in the software and tag the patients with different surgical types. The software may automatically look into the record and tag patients who are older than 65 years old and have low bone density. The surgeon may be able to ask the software to find candidates that are similar or match certain criteria of the patients in the category. This may help the researcher find patients for doing studies such as reviews on surgical techniques. The software may offer a way to export and analyze the data to aid in the study of the report.

In some embodiments, the digital interface may allow providers to track statistics and metrics of their choosing. These may be statistics of patient demographic, intervention types and frequencies, cases done, patients seen, time with patients, time planning interventions, number of devices utilized, or other metrics relevant to the system and the data collected.

In some embodiments, the digital system may enable the sharing of data to other parties. This may be the whole set of data collected or a limited set. This data may be identifiable or de-identified. In some embodiments the sharing may be between one or more providers enabling access to records and data for the patient. This may be used to plan and collaborate on treatment and assessment of patients.

According to some embodiments, by way of a non-limiting example, the digital system may be used by a MedTech company wanting to track the functional status and outcomes of patients with their new implant system. Health care providers may grant access to a set of patients categorized or tagged with the new implant system. This data may follow the patient from the very first assessment of the patient on the onset of treatment, through treatment, and after treatment. It may have multiple assessments. The MedTech company may evaluate the effect of their technology in comparable cases and evaluate patient selection. The data may be input into a model to enable better patient selection. The data may also be used to improve the product, make new products, or enable custom products based on the data collected. The company may segment populations based on the data from the system in order to select optimal implant matches for the patient based on the data collected.

In some embodiments, the outputs may be used in the diagnosis or treatment planning of a patient. By way of a non-limiting example, in some embodiments, a doctor may have a certain display to help differentiate diagnosis or to better understand the patient's condition. In some embodiments, a different display may be shown to the patient during the collection of the data to best collect information and to further instruct the patient. In some embodiments, after some data has been collected, another display may be shown to the patient to communicate their condition and to help educate them on their potential avenues for treatment.

According to some embodiments, the disclosed systems and methods may be utilized to provide live monitoring of individuals (see, e.g., Step 410, supra). In some embodiments, such monitoring may be used in settings such as rehabilitation, clinic visits, during gait analysis tests, walking tests, or incorporated into a mixed reality headset. In some embodiments, outputs of the UE may be modulated to focus on specific situations. In some embodiments, such modulations may include speed of walking, gait metrics, joint angles, physiological parameters, regression of disease states, muscular compensation, muscular activity, and fatigue through an activity.

In some embodiments, the disclosed sensor system and outputs may be combined into mixed reality systems in order to create clinical tests for patients. In some embodiments, the display may be presented to patients in order to create obstacles and tests in order to measure their response rates. In some embodiments, this may be paired with treadmill tests, standard clinical walk tests, and diagnostics.

In some embodiments, the disclosed sensor system may be expanded on alternate anatomical positions to assist with diagnostics, progression tracking, and treatment of diseases. In some embodiments, these sensors may be positioned around and/or on joints and extremities such as shoulders, hips, knees, ankles, wrists, and fingers.

In some embodiments, less (or a predetermined number of) sensors may be placed along the extremities in order to monitor recovery from procedures, such as total knee arthroplasties, total hip replacements; disease progression, such as carpal tunnel, rotator cuff injuries, arthritis; or treatment of diseases through electrical stimulation. In some embodiments, these sensors may be used in clinical visits, for at home monitoring, for in-patient monitoring, for post-surgical recovery monitoring, or for pre-operative monitoring of severity of lower and upper extremity conditions.

In some embodiments, the disclosed sensor system may create neurostimulation as a form of treatment for diseases. In some embodiments, such component of the disclosed embodiments may be modular such that it can be enabled or disabled by a physician or patient. In some embodiments related to neurostimulation, the user may interface with the engine 200 in order to alter the level of current, voltage, frequency of stimulations, and the like. In some embodiments, such alteration may be with regard to a specific level of a disease. In some embodiments, the neurostimulation may be trained through an AI/ML algorithm incorporating deep learning in order alter treatments throughout the course of treatment and recovery.

In some embodiments, engine 200 may be used to facilitate communication with practitioners, patients, athletes, trainers, and other individuals through telemedicine. In some embodiments, this may utilize any type of UE as discussed above—for example, an IoT device, tablet, iPad, phone, watch, tv, computer, or system connected through any type of network—such as, for example, WiFi, Bluetooth, data, or ethernet transmission. In some embodiments, this can involve a connection between/via a user wearing the sensors with an individual. In some embodiments, this individual may be conversing with the user instructing them through a series of steps, watching their performance, and/or monitoring their actions. In some embodiments, this individual may have access to live and/or recorded analysis from the sensors and algorithms, physiological parameters, and other information from the sensors. In some embodiments, this may be modulated in order to focus on specific parts of the body or specific actions. In some embodiments, a replay of the actions may be incorporated into the action in order to ease communication.

In some embodiments, the wearer or the user may be an animal. According to some embodiments, by way of a non-limiting example, a dog may wear this after orthopedic surgery to monitor motion and warn the owner of overuse or overloading. The system may be a video analysis or the full sensor array. The system may report the data back to the treating vet or rehab specialist treating the dog

In some embodiments, teleconferences may be used to assist with setting up the sensors and calibrating the devices. In some embodiments, the sensors may be re-prescribed to patients to facilitate telemedicine calls longitudinally throughout their care for progressive diseases.

Accordingly, as discussed herein, the disclosed technology provides novel capabilities and functionality for, via the sleek configuration of the disclosed sensor array, enabling an agile assessment of a patient's mobility, flexibility, pain, posture, and motion. As mentioned above, such functional assessments are useful for many aspects of medicine.

According to some embodiments, further disclosure of configurations, implementations and functionalities of the disclosed systems and methods are provided in the depictions of FIGS. 4B-4L (e.g., as originally disclosed in APPENDIX A of U.S. Provisional Application No. 63/442,984, which is incorporated herein by reference, discussed supra).

According to some embodiments, in FIG. 4B depicted are non-limited examples of positions of sensors on a skeleton with specific skeletal/vertebral positions In some embodiments, sensor configurations can vary from location to location, and additional placements may be added to the front of the chest, lower limbs, and/or any other body part or extremity.

In FIG. 4C, in some embodiments, provides a schematic depicting portions of where certain sensors can be placed in accordance with positions on a patient.

In FIG. 4D, in some embodiments, depicted are non-limited example embodiments for a chip design and features for a type of design specification for which the disclosed systems and methods, and corresponding sensing and patient monitoring, can be implemented. For example, the depicted sensor can be a form-factor for behind a patient's ear, as depicted in FIG. 4D.

In FIG. 4E, in some embodiments, depicted is a non-limiting example of how positional tracking via a UE can be performed and displayed within an interface(s), according to some embodiments of the present disclosure. Accordingly, in some embodiments, as depicted in FIG. 4F, depicted are additional interfaces and operational steps for performing the disclosed tracking.

In FIGS. 4G-4L, in some embodiments, depicted are non-limiting examples of the generated reports and computed data associated therewith, as per the processing of the steps of Process 400 (e.g., Steps 416-418, for example), discussed supra.

FIG. 7 is a schematic diagram illustrating a client device showing an example embodiment of a client device that may be used within the present disclosure. Client device 700 may include many more or fewer components than those shown in FIG. 7. However, the components shown are sufficient to disclose an illustrative embodiment for implementing the present disclosure. Client device 700 may represent, for example, UE 102 discussed above at least in relation to FIG. 1.

As shown in the figure, in some embodiments, Client device 700 includes a processing unit (CPU) 722 in communication with a mass memory 730 via a bus 724. Client device 700 also includes a power supply 726, one or more network interfaces 750, an audio interface 752, a display 754, a keypad 756, an illuminator 758, an input/output interface 760, a haptic interface 762, an optional global positioning systems (GPS) receiver 764 and a camera(s) or other optical, thermal or electromagnetic sensors 766. Device 700 can include one camera/sensor 766, or a plurality of cameras/sensors 766, as understood by those of skill in the art. Power supply 726 provides power to Client device 700.

Client device 700 may optionally communicate with a base station (not shown), or directly with another computing device. In some embodiments, network interface 750 is sometimes known as a transceiver, transceiving device, or network interface card (NIC).

Audio interface 752 is arranged to produce and receive audio signals such as the sound of a human voice in some embodiments. Display 754 may be a liquid crystal display (LCD), gas plasma, light emitting diode (LED), or any other type of display used with a computing device. Display 754 may also include a touch sensitive screen arranged to receive input from an object such as a stylus or a digit from a human hand.

Keypad 756 may include any input device arranged to receive input from a user. Illuminator 758 may provide a status indication and/or provide light.

Client device 700 also includes input/output interface 760 for communicating with external. Input/output interface 760 can utilize one or more communication technologies, such as USB, infrared, Bluetooth™, or the like in some embodiments. Haptic interface 762 is arranged to provide tactile feedback to a user of the client device.

Optional GPS transceiver 764 can determine the physical coordinates of Client device 700 on the surface of the Earth, which typically outputs a location as latitude and longitude values. GPS transceiver 764 can also employ other geo-positioning mechanisms, including, but not limited to, triangulation, assisted GPS (AGPS), E-OTD, CI, SAI, ETA, BSS or the like, to further determine the physical location of client device 700 on the surface of the Earth. In one embodiment, however, Client device may through other components, provide other information that may be employed to determine a physical location of the device, including for example, a MAC address, Internet Protocol (IP) address, or the like.

Mass memory 730 includes a RAM 732, a ROM 734, and other storage means. Mass memory 730 illustrates another example of computer storage media for storage of information such as computer readable instructions, data structures, program modules or other data. Mass memory 730 stores a basic input/output system (“BIOS”) 740 for controlling low-level operation of Client device 700. The mass memory also stores an operating system 741 for controlling the operation of Client device 700.

Memory 730 further includes one or more data stores, which can be utilized by Client device 700 to store, among other things, applications 742 and/or other information or data. For example, data stores may be employed to store information that describes various capabilities of Client device 700. The information may then be provided to another device based on any of a variety of events, including being sent as part of a header (e.g., index file of the HLS stream) during a communication, sent upon request, or the like. At least a portion of the capability information may also be stored on a disk drive or other storage medium (not shown) within Client device 700.

Applications 742 may include computer executable instructions which, when executed by Client device 700, transmit, receive, and/or otherwise process audio, video, images, and enable telecommunication with a server and/or another user of another client device. Applications 742 may further include a client that is configured to send, to receive, and/or to otherwise process gaming, goods/services and/or other forms of data, messages and content hosted and provided by the platform associated with engine 200 and its affiliates.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, and the like).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

For the purposes of this disclosure a module is a software, hardware, or firmware (or combinations thereof) system, process or functionality, or component thereof, that performs or facilitates the processes, features, and/or functions described herein (with or without human interaction or augmentation). A module can include sub-modules. Software components of a module may be stored on a computer readable medium for execution by a processor. Modules may be integral to one or more servers, or be loaded and executed by one or more servers. One or more modules may be grouped into an engine or an application.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores,” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, and the like).

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

For the purposes of this disclosure the term “user,” “subscriber” “consumer” or “customer” should be understood to refer to a user of an application or applications as described herein and/or a consumer of data supplied by a data provider. By way of example, and not limitation, the term “user” or “subscriber” can refer to a person who receives data provided by the data or service provider over the Internet in a browser session, or can refer to an automated software application which receives the data and stores or processes the data. Those skilled in the art will recognize that the methods and systems of the present disclosure may be implemented in many manners and as such are not to be limited by the foregoing exemplary embodiments and examples. In other words, functional elements being performed by single or multiple components, in various combinations of hardware and software or firmware, and individual functions, may be distributed among software applications at either the client level or server level or both. In this regard, any number of the features of the different embodiments described herein may be combined into single or multiple embodiments, and alternate embodiments having fewer than, or more than, all of the features described herein are possible.

Functionality may also be, in whole or in part, distributed among multiple components, in manners now known or to become known. Thus, myriad software/hardware/firmware combinations are possible in achieving the functions, features, interfaces and preferences described herein. Moreover, the scope of the present disclosure covers conventionally known manners for carrying out the described features and functions and interfaces, as well as those variations and modifications that may be made to the hardware or software or firmware components described herein as would be understood by those skilled in the art now and hereafter.

Furthermore, the embodiments of methods presented and described as flowcharts in this disclosure are provided by way of example in order to provide a more complete understanding of the technology. The disclosed methods are not limited to the operations and logical flow presented herein. Alternative embodiments are contemplated in which the order of the various operations is altered and in which sub-operations described as being part of a larger operation are performed independently.

While various embodiments have been described for purposes of this disclosure, such embodiments should not be deemed to limit the teaching of this disclosure to those embodiments. Various changes and modifications may be made to the elements and operations described above to obtain a result that remains within the scope of the systems and processes described in this disclosure.

What is claimed is:

1. A method comprising:
identifying, by a device, a set of locations corresponding to a body part of a user, each of the locations of the set of location having a physically associated sensor array;
executing, by the device, a collection instruction according to a predetermined time period, the executed collection instruction causing each sensor array to commence collecting data related to movements and motions of the user in relation to a respective location within the set of locations of the body part of the user, the collection of data being performed for a duration of the predetermined time period;
analyzing, by the device executing an artificial intelligence (AI) model, the collected data and determining, based on the AI-based analysis, metrics corresponding to a current status of the user; and
generating, by the device, an electronic clinical report based on the determined metrics, the electronic clinical report configured to visually display the determined metrics in a manner that depicts the status of the user in accordance with the body part based on the collected data related to the movements and motions.

2. The method of claim 1, further comprising:
calibrating the sensor array, the calibration comprising compiling the collection instruction according to an initial iteration prior to an iteration of the collection instruction.

3. The method of claim 2, wherein the calibration is performed based on the execution of a pose estimation algorithm, wherein the calibration is based on information related to the user corresponding to at least one of height, leg length, joint angles or positions, spinal segment angles, vertebral body angles, and bone lengths or angles.

4. The method of claim 1, further comprising:
collecting information related to the user, the user information corresponding to demographic and behavior information of the user; and
analyzing the collected data based in part on the collected user information, wherein the determination of metrics is based further on the user information.

5. The method of claim 4, further comprising:
analyzing the user information via an executed large language model (LLM); and
determining additional information related to at least one of a medical diagnosis, initial symptoms and initial observations related to the user, wherein the additional information is further utilized to determine the metrics of the user.

6. The method of claim 1, further comprising:
determining, based on the determined metrics, a health score;
determining a type of clinic report to provide based on the determined health score and a type of the body part, wherein the generation of the electronic clinical report is based on the determine type.

7. The method of claim 1, wherein the status of the patent, based on the determined metrics, correspond to at least one of posture, pain, movement, motion, treatment progress, mobility, flexibility and posture.

8. The method of claim 1, further comprising:
enabling, based on the identification of the set of locations, placement of each sensor array.

9. The method of claim 1, wherein the set of locations corresponds to a plurality of body parts, wherein the clinical report is based on collected data related to the plurality of body parts.

10. The method of claim 1, wherein the body part is selected from at least one body part of a human, wherein the body part comprises at least one of a spine and legs of the user, wherein the sensor arrays correspond to a type of the body part.

11. A system comprising:
a processor configured to:
identify a set of locations corresponding to a body part of a user, each of the locations of the set of location having a physically associated sensor array;
execute, a collection instruction according to a predetermined time period, the executed collection instruction causing each sensor array to commence collecting data related to movements and motions of the user in relation to a respective location within the set of locations of the body part of the user, the collection of data being performed for a duration of the predetermined time period;
analyze, via execution of an artificial intelligence (AI) model, the collected data and determine, based on the AI-based analysis, metrics corresponding to a current status of the user; and
generate an electronic clinical report based on the determined metrics, the electronic clinical report configured to visually display the determined metrics in a manner that depicts the status of the user in accordance with the body part based on the collected data related to the movements and motions.

12. The system of claim 11, wherein the processor is further configured to:
calibrate the sensor array, the calibration comprising compiling the collection instruction according to an initial iteration prior to an iteration of the collection instruction, wherein the calibration is performed based on the execution of a pose estimation algorithm, wherein the calibration is based on information related to the user corresponding to at least one of height, leg length, joint angles or positions, spinal segment angles, vertebral body angles, and bone lengths or angles.

13. The system of claim 11, wherein the processor is further configured to:
collect information related to the user, the user information corresponding to demographic and behavior information of the user; and
analyze the collected data based in part on the collected user information, wherein the determination of metrics is based further on the user information.

14. The system of claim 13, wherein the processor is further configured to:
analyze the user information via an executed large language model (LLM); and
determine additional information related to at least one of a medical diagnosis, initial symptoms and initial observations related to the user, wherein the additional information is further utilized to determine the metrics of the user.

15. The system of claim 14, wherein the processor is further configured to:
determine, based on the determined metrics, a health score;
determine a type of clinic report to provide based on the determined health score and a type of the body part, wherein the generation of the electronic clinical report is based on the determine type.

16. A non-transitory computer-readable storage medium tangibly encoded with computer-executable instructions that when executed by a processor, performs a method comprising:
identifying, by the processor, a set of locations corresponding to a body part of a user, each of the locations of the set of location having a physically associated sensor array;
executing, by the device, a collection instruction according to a predetermined time period, the executed collection instruction causing each sensor array to commence collecting data related to movements and motions of the user in relation to a respective location within the set of locations of the body part of the user, the collection of data being performed for a duration of the predetermined time period;
analyzing, by the device executing an artificial intelligence (AI) model, the collected data and determining, based on the AI-based analysis, metrics corresponding to a current status of the user; and
generating, by the device, an electronic clinical report based on the determined metrics, the electronic clinical report configured to visually display the determined metrics in a manner that depicts the status of the user in accordance with the body part based on the collected data related to the movements and motions.

17. The non-transitory computer-readable storage medium of claim 16, further comprising:
calibrating the sensor array, the calibration comprising compiling the collection instruction according to an initial iteration prior to an iteration of the collection instruction, wherein the calibration is performed based on the execution of a pose estimation algorithm, wherein the calibration is based on information related to the user corresponding to at least one of height, leg length, joint angles or positions, spinal segment angles, vertebral body angles, and bone lengths or angles.

18. The non-transitory computer-readable storage medium of claim 16, further comprising:
collecting information related to the user, the user information corresponding to demographic and behavior information of the user; and
analyzing the collected data based in part on the collected user information, wherein the determination of metrics is based further on the user information.

19. The non-transitory computer-readable storage medium of claim 18, further comprising:
analyzing the user information via an executed large language model (LLM); and
determining additional information related to at least one of a medical diagnosis, initial symptoms and initial observations related to the user, wherein the additional information is further utilized to determine the metrics of the user.

20. The non-transitory computer-readable storage medium of claim 19, further comprising:
determining, based on the determined metrics, a health score;
determining a type of clinic report to provide based on the determined health score and a type of the body part, wherein the generation of the electronic clinical report is based on the determine type.
